(19) United States
(12) Patent Application Publication
(10) Pub. No.: US 2014/0065234 A1
(43) Pub. Date: Mar. 6, 2014

(54) POLYMERSOMES, LIPOSOMES, AND OTHER SPECIES ASSOCIATED WITH FLUIDIC DROPLETS

(71) Applicants: Ho Cheung Shum, Hong Kong (CN); Daeyeon Lee, Wynnewood, PA (US); Insun Yoon, Belmont, MA (US); David A. Weitz, Bolton, MA (US); Jin-Woong Kim, Gyeonggi-do (KR)

(72) Inventors: Ho Cheung Shum, Hong Kong (CN); Daeyeon Lee, Wynnewood, PA (US); Insun Yoon, Belmont, MA (US); David A. Weitz, Bolton, MA (US); Jin-Woong Kim, Gyeonggi-do (KR)

(73) Assignee: President and Fellows of Harvard College, Cambridge, MA (US)

(21) Appl. No.: 13/967,018

(22) Filed: Aug. 14, 2013

Related U.S. Application Data
(63) Continuation of application No. 12/993,205, filed on Mar. 16, 2011, now abandoned.
(60) Provisional application No. 61/059,163, filed on Jun. 5, 2008.

(51) Int. Cl. A61K 47/34; A61K 9/16

(52) U.S. Cl. CPC A61K 47/34; A61K 9/16
USPC 424/501; 525/437; 525/434; 514/772.1

(57) ABSTRACT

The present invention relates generally to vesicles such as liposomes, colloidosomes, and polymersomes, as well as techniques for making and using such vesicles. In some cases, the vesicles may be at least partially biocompatible and/or biodegradable. The vesicles may be formed, according to one aspect, by forming a multiple emulsion comprising a first droplet surrounded by a second droplet, which in turn is surrounded by a third fluid, where the second droplet comprises lipids and/or polymers, and removing fluid from the second droplet, e.g., through evaporation or diffusion, until a vesicle is formed. In certain aspects, the size of the vesicle may be controlled, e.g., through osmolarity, and in certain embodiments, the vesicle may be ruptured through a change in osmolarity. In some cases, the vesicle may contain other species, such as fluorescent molecules, microparticles, pharmaceutical agents, etc., which may be released upon rupture. Yet other aspects of the invention are generally directed to methods of making such vesicles, kits involving such vesicles, or the like.

### RELATED APPLICATIONS

This application is a continuation of U.S. application Ser. No. 12/993,205, filed Nov. 17, 2010 which is a U.S. National Stage Application of International Application No.: PCT/US2009/003389, filed Jun. 4, 2009 which claims the benefit of U.S. Provisional Patent Application Ser. No. 61/059,163, filed Jun. 5, 2008, entitled “Polymersomes, Liposomes, and other Species Associated with Fluidic Droplets,” by Shum, et al., incorporated herein by reference.

### GOVERNMENT FUNDING

Research leading to various aspects of the present invention were sponsored, at least in part, by the National Science Foundation under Grant Nos. DMR-0213805 and DMR-0602684. The U.S. Government has certain rights in the invention.

### FIELD OF INVENTION

The present invention relates generally to vesicles such as liposomes, colloidosomes, and polymersomes, as well as techniques for making and using such vesicles. In some cases, the vesicles may be at least partially biocompatible and/or biodegradable.

### BACKGROUND

Vesicles such as liposomes and polymersomes can be described as having a membrane or an outer layer surrounding an inner fluid. The membrane can include lipids (as in a liposome) and/or polymers (as in a polymersome). The fluids within the vesicle and outside the vesicle may be the same or different. Examples of liposomes include those formed from naturally-derived phospholipids with mixed lipid chains (like egg phosphatidylethanolamine), or pure surfactant components like DOPE (dioleoylphosphatidylethanolamine). Examples of polymersomes include those described in International Patent Application No. PCT/US2006/007772, filed Mar. 3, 2006, entitled “Method and Apparatus for Forming Multiple Emulsions,” by Weitz, et al., published as WO 2006/096571 on Sep. 14, 2006, incorporated herein by reference.

### SUMMARY OF THE INVENTION

The present invention relates generally to vesicles such as liposomes, colloidosomes, and polymersomes, as well as techniques for making and using such vesicles. In some cases, the vesicles may be at least partially biocompatible and/or biodegradable. The subject matter of the present invention involves, in some cases, interrelated products, alternative solutions to a particular problem, and/or a plurality of different uses of one or more systems and/or articles.

In one aspect, the present invention is directed to an article. The article, according to one set of embodiments, includes a polymersome comprising a multiblock copolymer. In some cases, at least one of the blocks of the copolymer is a biodegradable polymer.

Another aspect of the present invention is generally directed to a method. The method, according to one set of embodiments, includes acts of forming a first droplet from a first fluid stream surrounded by a second fluid while the second fluid is surrounded by a third fluid, and reducing the amount of the second fluid in the second fluid droplet. In some instances, the second fluid contains a biodegradable polymer.

In another set of embodiments, the method includes acts of providing a polymersome comprising a diblock or a triblock copolymer, and exposing the polymersome to a change in osmolarity at least sufficient to cause the polymersome to rupture. In some embodiments, at least one of the blocks of the copolymer is a biodegradable polymer.

In another aspect, the present invention is directed to a method of making one or more of the embodiments described herein, for example, a polymersome that is at least partially biocompatible or biodegradable. In another aspect, the present invention is directed to a method of using one or more of the embodiments described herein, for example, a polymersome that is at least partially biocompatible or biodegradable.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control. If two or more documents incorporated by reference include conflicting and/or inconsistent disclosure with respect to each other, then the document having the later effective date shall control.

### BRIEF DESCRIPTION OF THE DRAWINGS

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIG. 1 is a schematic illustration of a microfluidic device useful in making multiple emulsions, in one embodiment of the invention;

FIG. 2 illustrates the formation of a polymersome, according to another embodiment of the invention;

FIG. 3 illustrates another microfluidic device useful in making multiple emulsions, in yet another embodiment of the invention;

FIGS. 4A-4J illustrate a double emulsion drop undergoing dewetting, in one embodiment of the invention;

FIG. 5 is a schematic diagram showing a proposed structure of a double emulsion drop;

FIGS. 6A-6C illustrate various polymersomes formed in certain embodiments of the invention;

FIGS. 7A-7L illustrate the shrinkage and rupture of a polymersome due to osmotic shock, in another embodiment of the invention;

FIGS. 8A-8I illustrate certain polymersomes formed in various embodiments of the invention;

FIGS. 9A-9D illustrate the use of a homopolymer to stabilize a double emulsion, in one embodiment of the invention;

FIG. 10 illustrates the formation of a phospholipid vesicle, according to one embodiment of the invention;

FIGS. 11A-11C illustrate certain phospholipid double emulsions, in another embodiment of the invention;

FIGS. 12A-12F illustrates vesicle formation, in yet another embodiment of the invention;

FIGS. 13A-13B illustrate various liposomes of certain embodiments of the invention;

FIGS. 14A-14B illustrate certain vesicles containing microspheres, in another embodiment of the invention;

FIGS. 15A-15D illustrate shocked polyemrsomes, in one embodiment of the invention;

FIGS. 16A-16C illustrate buckled polymersomes, in another embodiment of the invention;

FIGS. 17A-17D illustrate a microfluidic technique useful for producing nanoparticle colloidosomes, in one embodiment of the invention;

FIGS. 18A-18D illustrate the effects of flow rates on various double emulsions, in another embodiment of the invention;

FIGS. 19A-19D illustrate SEM images of various nanoparticle colloidosomes, in accordance with other embodiments of the invention;

FIGS. 20A-20C illustrate confocal laser scanning microscope images of nanoparticle colloidosomes, in still other embodiments of the invention;

FIG. 21 illustrates FRAP data of a nanoparticle colloidosomes, in yet another embodiment of the invention;

FIG. 22A-22F illustrates various double emulsions, in still another embodiment of the invention;

FIG. 23A is an optical microscopy image of colloidosomes suspended in water, in another embodiment of the invention;

FIG. 23B is a high magnification freeze-fracture cryo-SEM image of a colloidosomes shell, in still another embodiment of the invention;

FIGS. 24A-24D illustrate the formation of polymersomes in various solvents, in accordance with one embodiment of the invention;

FIG. 25 illustrates various multi-compartment polymersomes, in accordance with another embodiment of the invention;

FIGS. 26A-26C illustrate optical micrographs of various polymersomes, in yet another embodiment of the invention; and

FIGS. 27A-27B illustrate various labeled polymersomes, in still another embodiment of the invention.

### DETAILED DESCRIPTION

The present invention relates generally to vesicles such as liposomes, colloidosomes, and polymersomes, as well as techniques for making and using such vesicles. In some cases, the vesicles may be at least partially biocompatible and/or biodegradable. The vesicles may be formed, according to one aspect, by forming a multiple emulsion comprising a first droplet surrounded by a second droplet, which in turn is surrounded by a third fluid, where the second droplet comprises lipids and/or polymers, and removing fluid from the second droplet, e.g., through evaporation or diffusion, until a vesicle is formed. In certain aspects, the size of the vesicle may be controlled, e.g., through osmolarity, and in certain embodiments, the vesicle may be ruptured through a change in osmolarity. In some cases, the vesicle may contain other species, such as fluorescent molecules, microparticles, pharmaceutical agents, etc., which may be released upon rupture. Yet other aspects of the invention are generally directed to methods of making such vesicles, kits involving such vesicles, or the like.

As discussed above, a vesicle can be described as having a membrane or a “shell” surrounding an inner fluid. The membrane (not necessarily solid) may include lipids (i.e., a liposome), polymers (i.e., a polymersome or a polymerosome), and/or colloidal particles (i.e., a colloidosome). In some cases, more than one of these may be present. For example, a vesicle may be both a liposome and a colloidosome, a liposome and a polymersome, a colloidosomes and a polymersome, etc. The polymer may be, for instance, diblock or a triblock copolymer, which can be amphiphilic; examples of such polymers are discussed below. In some cases, where block copolymers, homopolymers may also be used (e.g., having the same composition as one of the blocks of the copolymer), e.g., to stabilize the vesicle. A “block copolymer” is given its usual definition in the field of polymer chemistry. A block is typically a portion of a polymer comprising a series of repeat units that are distinguishable from adjacent portions of the block. Thus, for instance, a diblock copolymer comprises a first repeat unit and a second repeat unit; a triblock copolymer includes a first repeat unit, a second repeat unit, and a third repeat unit; a multiblock copolymer includes a plurality of such repeat units, etc. As a specific example, a diblock copolymer may comprise a first portion defined by a first repeat unit and a second portion defined by a second repeat unit; in some cases, the diblock copolymer may further comprise a third portion defined by the first repeat unit (e.g., arranged such that the first and third portions are separated by the second portion), and/or additional portions defined by the first and second repeat units.

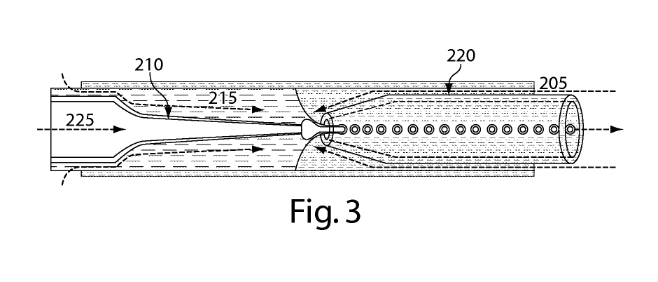

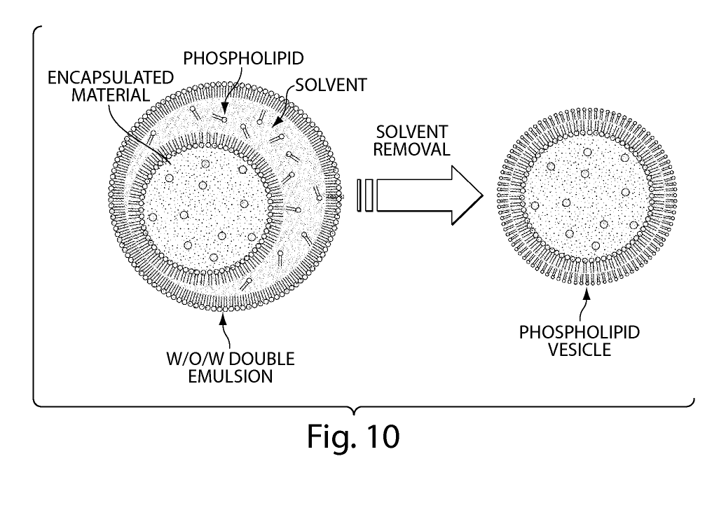

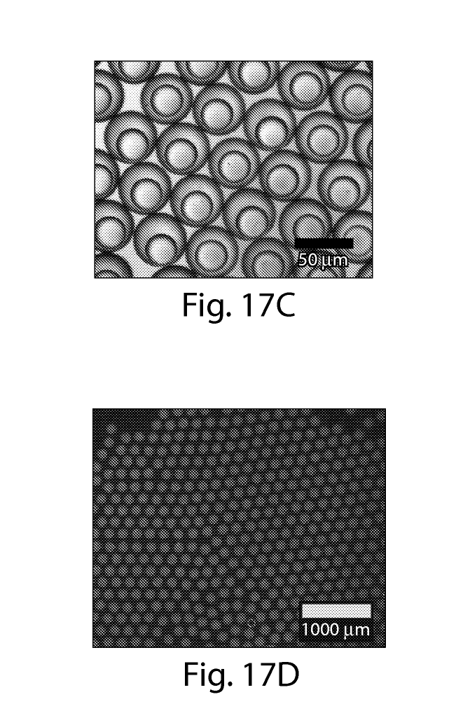

In some cases, a vesicle may include both lipids, polymers, and/or particles in its membrane. The membrane of the vesicle is typically a bilayer of lipids and/or polymers, e.g., as shown in FIG. 2 or FIG. 10. In some cases, however, the vesicle may include more than one membrane. In certain embodiments, the vesicle may include particles, e.g., as shown in FIG. 17B.

Fields in which vesicles may prove useful include, for example, food, beverage, health and beauty aids, paints and coatings, chemical separations, and drugs and drug delivery. For instance, a precise quantity of a drug, pharmaceutical, or other agent can be contained within a vesicle designed to release its contents under particular conditions, such as changes in osmolarity, as described in detail below, or the vesicle may be induced to join a cell, e.g., by fusing to the cell lipid bilayer. In some instances, cells can be contained within a vesicle, and the cells can be stored and/or delivered. Other species that can be stored and/or delivered include, for example, biochemical species such as nucleic acids such as siRNA, RNAi and DNA, proteins, peptides, or enzymes. Additional species that can be incorporated within a vesicle of the invention include, but are not limited to, microparticles, nanoparticles, quantum dots, fragrances, proteins, indicators, dyes, fluorescent species, chemicals, drugs, vitamins, growth factors, or the like. A vesicle can also serve as a reaction vessel in certain cases, such as for controlling chemical reactions.

Using the methods and devices described herein, in some embodiments, a consistent size and/or number of vesicles can be produced. For example, in some cases, a vesicle of a predictable size can be used to contain a specific quantity of a drug. In addition, combinations of compounds or drugs may be stored, transported, or delivered in a vesicle. For instance, hydrophobic and hydrophilic species can be delivered in a single vesicle, as it can include both hydrophilic and hydrophobic portions. The amount and concentration of each of these portions can be consistently controlled in a vesicle according to certain embodiments of the invention, which can provide for a predictable and consistent ratio of two or more species.

In one aspect of the invention, vesicles can be formed that can include lipids (e.g., as in a liposome) and/or polymers (e.g., as in a polymersome) and/or particles (e.g., as in a colloidosome). Vesicles such as polymersomes, colloidosomes, or liposomes may be formed, for example, using multiple emulsion techniques such as those described below. Non-limiting examples of polymers that can be used include normal butyl acrylate and acrylic acid, which can be polymerized to form a copolymer of poly(normal-butyl acrylate)-poly(acrylic acid); poly(ethylene glycol) and poly(lactic acid), which can be polymerized to form a copolymer of poly(ethylene glycol)-poly(lactic acid); or poly(ethylene glycol) and poly(glycolic acid), which can be polymerized to form a copolymer of poly(ethylene glycol)-poly(glycolic acid). In some cases, the copolymer may comprise more than two types of monomers, for example, as in a copolymer of poly(ethylene glycol)-poly(lactic acid)-poly(glycolic acid). The monomers may be distributed in any suitable order within the copolymer, for example, as separate blocks (e.g., a multiblock copolymer), randomly, alternating, etc. “Polymers,” as used herein, may include polymeric compounds, as well as compounds and species that can form polymeric compounds, such as prepolymers. Prepolymers include, for example, monomers and oligomers. In some cases, however, only polymeric compounds are used and prepolymers may not be appropriate.

Examples of biodegradable or biocompatible polymers include, but are not limited to, poly(lactic acid), poly(glycolic acid), polyanhydride, poly(caprolactone), poly(ethylene oxide), polybutylene terephthalate, starch, cellulose, chitosan, and/or combinations of these. A “biodegradable material,” as used herein, is a material that will degrade in the presence of physiological solutions (which can be mimicked using phosphate-buffered saline) on the time scale of days, weeks, or months (i.e., its half-life of degradation can be measured on such time scales). As used herein, “biocompatible” is given its ordinary meaning in the art. For instance, a biocompatible material may be one that is suitable for implantation into a subject without adverse consequences, for example, without substantial acute or chronic inflammatory response and/or acute rejection of the material by the immune system, for instance, via a T-cell response. It will be recognized, of course, that “biocompatibility” is a relative term, and some degree of inflammatory and/or immune response is to be expected even for materials that are highly biocompatible. However, non-biocompatible materials are typically those materials that are highly inflammatory and/or are acutely rejected by the immune system, i.e., a non-biocompatible material implanted into a subject may provoke an immune response in the subject that is severe enough such that the rejection of the material by the immune system cannot be adequately controlled, in some cases even with the use of immunosuppressant drugs, and often can be of a degree such that the material must be removed from the subject. In some cases, even if the material is not removed, the immune response by the subject is of such a degree that the material ceases to function; for example, the inflammatory and/or the immune response of the subject may create a fibrous “capsule” surrounding the material that effectively isolates it from the rest of the subject's body; materials eliciting such a reaction would also not be considered as “biocompatible.”

Non-limiting examples of lipids that can be used in a vesicle include saturated (e.g., DPPC, DMPC, or DSPC) and/or unsaturated (e.g., DOPC or POPC) phosphocholines used alone or mixed with a phospho-L-serine (DPPS). These abbreviations are as follows: DPPC: 1,2-dipalmitoyl-sn-glycero-3-phosphocholine; DMPC: 1,2-dimyristoyl-sn-glycero-3-phosphocholine; DSPC: 1,2-distearoyl-sn-glycero-3-phosphocholine; DOPC: 1,2-dioleoyl-sn-glycero-3-phosphocholine; POPC: 1-palmitoyl-2-oleoyl-sn-glyceo-3-phoscholine; DPPS: 1,2-diacyl-sn-glycero-3-phospho-L-serine.

Any suitable particles may be used in a colloidosome, including hydrophilic and/or hydrophobic particles. Examples of hydrophobic materials which may be used to form the particles include polystyrene, polyalkylmethacrylates, such as polymethylmethacrylate, polyethylmethyacrylate, polybutylmethacrylate; polyalkylenes, including polyethylene and polypropylene; and inorganic materials such as ceramics and including silica, alumina, titania that are surface-functionalized to make them hydrophobic. In some cases, some of eth particles may be magnetic. Suitable hydrophilic materials which can be used to form the particles include organic polymers that can be functionalized with hydrophilic groups; clay particles, such as disk-shaped particles; biological materials, including pollen grains, seeds, and virus particles that have been treated so as to be non-infective or to otherwise to not cause disease; and particles, including nanoparticles, composed of metallic, electrically semiconducting or insulating materials, including gold, cadmium sulfide, cadmium selenide, zinc sulfate and combinations thereof.

In some cases, the particles may be nanoparticles, e.g., having an average diameter of less than about 1 micrometer. The average diameter of a nonspherical particle is the diameter of a perfect sphere having the same volume as the particle. In some cases, the average diameters of the particles may be, for example, less than about 1 micrometer, less than about 500 nm, less than about 200 nm, less than about 100 nm, less than about 75 nm, less than about 50 nm, less than about 25 nm, less than about 20 nm, less than about 10 nm, or less than about 5 nm in some cases. The average diameter may also be at least about 1 micrometer, at least about 2 nm, at least about 3 nm, at least about 5 nm, at least about 10 nm, at least about 15 nm, or at least about 20 nm in certain cases.

Other examples include those disclosed U.S. patent application Ser. No. 12/019,454, filed Jan. 24, 2008, entitled “Colloidosomes Having Tunable Properties and Methods for Making Colloidosomes Having Tunable Properties,” by Kim, et al., and U.S. patent application Ser. No. 10/433,753, filed Dec. 8, 2003, entitled “Methods and Compositions for Encapsulating Active Agents,” by Bausch, et al., published as U.S. Patent Application Publication No. 2004/0096515 on May 20, 2004, each incorporated herein by reference.

In some embodiments, a colloidosome may have relatively well-defined pores whose size can be varied depending on the application. For example, if a colloidosome has encapsulated therein a biological cell, the pores may be sized to be large enough to allow any desirable substance produced by the cell to diffuse out of the chamber through the pores and external to the colloidosome, as well as allow desirable substances necessary to sustain the cell, such as glucose or other nutrients, to enter the chamber. The pores may be selected for such an application to be sufficiently small or otherwise sized to prevent entry into the chamber by immune system cells or immune system components, such as various antibodies, and/or to prevent the encapsulated cell from exiting the chamber through the pores. As described herein, the pore size can be adjusted by the size of the particles utilized. For example, use of particles of larger diameter can lead to larger pore sizes whereas use of beads of smaller diameter can lead to smaller pore sizes. Although pore size can vary depending on the application, non-limiting examples of pore sizes range from about 3 nm to about 3 micrometers, about 10 nm to about 1000 nm, or about 75 nm to about 200 nm, etc. When encapsulating a biological cell, pore sizes may be selected to be no more than about 1 micrometer to about 3 micrometers.

In certain embodiments of the invention, the pore sizes in a colloidosome are substantially uniform. That is, at least about 90%, or about 95%, or even about 100% of the pores of the colloidosome are of about the same size and may, for example, have the same average diameter, or vary no more than about 10%, about 5%, or about 2% of the average diameter of the pores within the colloidosome. The average diameter of a non-circular pore is the diameter of a circle having the same surface area as that of the pore. In other embodiments, the radius of the pores may differ by about 50% to about 300%, resulting in pores differing in diameter by up to a factor of about 1.5, or even by a factor up to about 4. In yet another embodiment, the pores may differ in radius by up to about 50%.

In some cases, the vesicle may include amphiphilic species such as amphiphilic polymers or lipids. The amphiphilic species typically includes a relatively hydrophilic portion, and a relatively hydrophobic portion. For instance, the hydrophilic portion may be a portion of the molecule that is charged, and the hydrophobic portion of the molecule may be a portion of the molecule that comprises hydrocarbon chains. Other amphiphilic species may also be used, besides diblock copolymers. For example, other polymers, or other species such as lipids or phospholipids may be used with the present invention.

Upon formation of a multiple emulsion or a vesicle, an amphiphilic species that is contained, dissolved, or suspended in the emulsion can spontaneously associate along a hydrophilic/hydrophobic interface in some cases. For instance, the hydrophilic portion of an amphiphilic species may extend into the aqueous phase and the hydrophobic portion may extend into the non-aqueous phase. Thus, the amphiphilic species can spontaneously organize under certain conditions so that the amphiphilic species molecules orient substantially parallel to each other and are oriented substantially perpendicular to the interface between two adjoining fluids, such as an inner droplet and outer droplet, or an outer droplet and an outer fluid. As the amphiphilic species become organized, they may form a sheet or a membrane, e.g., a substantially spherical sheet, with a hydrophobic surface and an opposed hydrophilic surface. Depending on the arrangement of fluids, the hydrophobic side may face inwardly or outwardly and the hydrophilic side may face inwardly or outwardly. The resulting structure may be a bilayer or a multi-lamellar structure.

In various aspects of the present invention, a vesicle may be made using multiple emulsions, such as those disclosed in U.S. patent application Ser. No. 11/885,306, filed Aug. 29, 2007, entitled “Method and Apparatus for Forming Multiple Emulsions,” by Weitz, et al.; or U.S. patent application Ser. No. 12/058,628, filed Mar. 28, 2008, entitled “Emulsions and Techniques for Formation,” by Chu, et al., each incorporated herein by reference. The multiple emulsions may be formed using any suitable process, for instance, those disclosed in U.S. Provisional Patent Application Ser. No. 61/160,020, filed Mar. 13, 2009, entitled “Controlled Creation of Multiple Emulsions,” by Weitz, et al., incorporated herein by reference. A multiple emulsion typically includes larger fluidic droplets that contain one or more smaller droplets therein which, in some cases, can contain even smaller droplets therein, etc. In some cases, the multiple emulsion is surrounded by a liquid (e.g., suspended). Any of these droplets may be of substantially the same shape and/or size (i.e., “monodisperse”), or of different shapes and/or sizes, depending on the particular application.

As used herein, the term “fluid” generally refers to a substance that tends to flow and to conform to the outline of its container, i.e., a liquid, a gas, a viscoelastic fluid, etc. Typically, fluids are materials that are unable to withstand a static shear stress, and when a shear stress is applied, the fluid experiences a continuing and permanent distortion. The fluid may have any suitable viscosity that permits flow. If two or more fluids are present, each fluid may be independently selected among essentially any fluids (liquids, gases, and the like) by those of ordinary skill in the art, by considering the relationship between the fluids. In some cases, the droplets may be contained within a carrier fluid, e.g., a liquid.

A “droplet,” as used herein, is an isolated portion of a first fluid that is surrounded by a second fluid. It is to be noted that a droplet is not necessarily spherical, but may assume other shapes as well, for example, depending on the external environment. In one embodiment, the droplet has a minimum cross-sectional dimension that is substantially equal to the largest dimension of the channel perpendicular to fluid flow in which the droplet is located. In some cases, the droplet may be a vesicle, such as a liposome, a colloidosome, or a polymersome.

In certain instances, the droplets may be contained within a carrying fluid, e.g., within a fluidic stream. The fluidic stream, in one set of embodiments, is created using a microfluidic system, discussed in detail below. In some cases, the droplets will have a homogenous distribution of diameters, i.e., the droplets may have a distribution of diameters such that no more than about 10%, about 5%, about 3%, about 1%, about 0.03%, or about 0.01% of the droplets have an average diameter greater than about 10%, about 5%, about 3%, about 1%, about 0.03%, or about 0.01% of the average diameter of the droplets. Techniques for producing such a homogenous distribution of diameters are also disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference, and in other references as described below.

The fluidic droplets may have any shape and/or size. Typically, monodisperse droplets are of substantially the same size. The shape and/or size of the fluidic droplets can be determined, for example, by measuring the average diameter or other characteristic dimension of the droplets. The “average diameter” of a plurality or series of droplets is the arithmetic average of the average diameters of each of the droplets. Those of ordinary skill in the art will be able to determine the average diameter (or other characteristic dimension) of a plurality or series of droplets, for example, using laser light scattering, microscopic examination, or other known techniques. The average diameter of a single droplet, in a non-spherical droplet, is the diameter of a perfect sphere having the same volume as the non-spherical droplet. The average diameter of a droplet (and/or of a plurality or series of droplets) may be, for example, less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. The average diameter may also be at least about 1 micrometer, at least about 2 micrometers, at least about 3 micrometers, at least about 5 micrometers, at least about 10 micrometers, at least about 15 micrometers, or at least about 20 micrometers in certain cases. In certain cases, the size of the vesicle may also be controlled by controlling the osmolarity of the solution surrounding the vesicle.

The multiple emulsions described herein may be made in a single step using different fluids. In one set of embodiments, a triple emulsion may be produced, i.e., an emulsion containing a first fluid, surrounded by a second fluid, which in turn is surrounded by a third fluid. In some cases, the third fluid and the first fluid may be the same, or the fluids may be substantially miscible. These fluids are often of varying miscibilities due to differences in hydrophobicity. For example, the inner fluid may be water soluble, the middle fluid oil soluble, and the outer fluid water soluble. This arrangement is often referred to as a w/o/w multiple emulsion (“water/oil/water”). Another multiple emulsion may include an inner fluid that is oil soluble, a middle fluid that is water soluble, and an outer fluid that is oil soluble. This type of multiple emulsion is often referred to as an o/w/o multiple emulsion (“oil/water/oil”). It should be noted that the term “oil” in the above terminology merely refers to a fluid that is generally more hydrophobic and not miscible in water, as is known in the art. Thus, the oil may be a hydrocarbon in some embodiments, but in other embodiments, the oil may comprise other hydrophobic fluids. More specifically, as used herein, two fluids are immiscible, or not miscible, with each other when one is not soluble in the other to a level of at least 10% by weight at the temperature and under the conditions at which the emulsion is produced. For instance, two fluids may be selected to be immiscible within the time frame of the formation of the fluidic droplets.

The fluids within the multiple emulsion droplet may the same, or different. The fluids may be chosen such that the inner droplets remain discrete, relative to their surroundings. As non-limiting examples, a fluidic droplet may be created having an outer droplet, containing one or more first fluidic droplets, some or all of which may contain one or more second fluidic droplets. In some cases, the outer fluid and the second fluid may be identical or substantially identical; however, in other cases, the outer fluid, the first fluid, and the second fluid may be chosen to be essentially mutually immiscible. One non-limiting example of a system involving three essentially mutually immiscible fluids is a silicone oil, a mineral oil, and an aqueous solution (i.e., water, or water containing one or more other species that are dissolved and/or suspended therein, for example, a salt solution, a saline solution, a suspension of water containing particles or cells, or the like). Another example of a system is a silicone oil, a fluorocarbon oil, and an aqueous solution. Yet another example of a system is a hydrocarbon oil (e.g., hexadecane), a fluorocarbon oil, and an aqueous solution. Non-limiting examples of suitable fluorocarbon oils include octadecafluorodecahydronaphthalene:

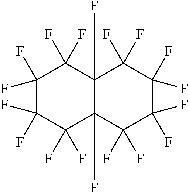 [SMILES: FC1(F)C(F)(F)C(F)(F)C2(F)C(F)(F)C(F)(F)C(F)(F)C(F)(F)C2(F)C1(F)F]

or 1-(1,2,2,3,3,4,4,5,5,6,6-undecafluorocyclohexyl)ethanol:

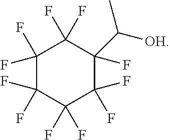 [SMILES: CC(O)C1(F)C(F)(F)C(F)(F)C(F)(F)C(F)(F)C1(F)F]

As fluid viscosity can affect droplet formation, in some cases the viscosity of any of the fluids in the fluidic droplets may be adjusted by adding or removing components, such as diluents, that can aid in adjusting viscosity. For example, in some embodiments, the viscosity of the outer fluid and the first fluid are equal or substantially equal. This may aid in, for example, an equivalent frequency or rate of droplet formation in the outer and fluid fluids. In other embodiments, the viscosity of the first fluid may be equal or substantially equal to the viscosity of the second fluid, and/or the viscosity of the outer fluid may be equal or substantially equal to the viscosity of the second fluid. In yet another embodiment, the outer fluid may exhibit a viscosity that is substantially different from either the first or second fluids. A substantial difference in viscosity means that the difference in viscosity between the two fluids can be measured on a statistically significant basis. Other distributions of fluid viscosities within the droplets are also possible. For example, the second fluid may have a viscosity greater than or less than the viscosity of the first fluid (i.e., the viscosities of the two fluids may be substantially different), the first fluid may have a viscosity that is greater than or less than the viscosity of the outer fluid, etc.

In one aspect, a vesicle such as a liposome, a colloidosome, or a polymersome may be formed by removing a portion of the middle fluid of a multiple emulsion. For instance, a component of the middle fluid, such as a solvent or carrier, can be removed from the fluid, in part or in whole, through evaporation or diffusion. As an example, in some cases, the middle fluid comprises a solvent system used as a carrier, and dissolved or suspended polymers or lipids, such as those described herein. After formation of a multiple emulsion, the solvent can be removed from the middle fluid using techniques such as evaporation or diffusion, leaving the polymers or lipids behind. For instance, as the solvent leaves the middle fluid layer, the polymers or lipids can self-assemble into single or multiple layers on the inner and/or outer surfaces, resulting in a vesicle such as a polymersome, colloido some, or a liposome. This can result in a thin layer of material that is capable of carrying, protecting, and delivering the inner droplet. Once formed, these vesicles can be removed from the outer fluid, dried, stored, etc. A specific example is given in FIG. 2, where a polymersome is formed from a multiple emulsion containing polymer. Other examples are given below.

In some cases, a component of the middle fluid may be removed through evaporation. In some cases, the evaporation rate of the component may be relatively slow. Without wishing to be bound by any theory, it is believed that relatively slow evaporation rates may reduce or inhibit destabilization or rupture during the evaporation process, for instance by reducing the stresses experienced by the vesicle during the evaporation process. For instance, the evaporation rate may be controlled such that between about 50% and about 90% of the middle fluid remains within the vesicle after about 1 day. In some cases, at least about 60%, at least about 70%, or at least about 80% of the middle fluid remains within the vesicle after about 1 day. The evaporation rate may be controlled, for instance, by using a loosely sealed container to slow the evaporation rate, by controlling the relatively humidity around the vesicles, by controlling the amount of airflow or exchange of gases that occurs around the vesicles, or the like.

In cases where it may be desirable to remove a portion of the middle fluid from the outer drop, some of the components of the middle fluid may be at least partially miscible in the outer fluid. This can allow the components to diffuse over time into the outer solvent, reducing the concentration of the components in the outer droplet, which can effectively increase the concentration of any of the immiscible components, e.g., polymers or surfactants, that comprise the outer droplet. This can lead to the self-assembly or gelation of the polymers, lipids, or other precursors in some embodiments, and can result in the formation of a vesicle having a solid or semi-solid shell. During droplet formation, it may still be preferred that the middle fluid be at least substantially immiscible with the outer fluid. This immiscibility can be provided, for example, by polymers, lipids, surfactants, solvents, or other components that form a portion of the middle fluid, but are not able to readily diffuse, at least entirely, into the outer fluid after droplet formation. Thus, the middle fluid can include, in certain embodiments, both a miscible component that can diffuse into the outer fluid after droplet formation, and an immiscible component that helps to promote droplet formation.

The remaining component or components of the middle fluid may self-organize as a result of the reduction in the amount of solvent or carrier in the middle fluid, for example, through crystallization or self-assembly of polymers or lipids dissolved in the middle fluid, e.g., to form a bilayer. For instance, polymers or lipids can be used so that when the concentration in the middle fluid increases (e.g., concurrently with a decrease in the solvent concentration) the molecules are oriented to form a membrane or a “shell” of lamellar sheets composed primarily or substantially of polymers or lipids. The membrane may be solid or semi-solid in some cases, e.g., forming a shell. For example, lipids and/or polymers within the membrane may be cross-linked to harden the membrane.

In some aspects, a vesicle such as a liposome, a colloidosome, or a polymersome may be caused to dissolve, rupture, or otherwise release its contents. Various species that can be contained within a fluidic droplet that can be released, for instance, pharmaceutical agents, nanoparticles, microparticles, drugs, DNA, RNA, proteins, fragrance, reactive agents, biocides, fungicides, preservatives, chemicals, cells, etc., as discussed herein.

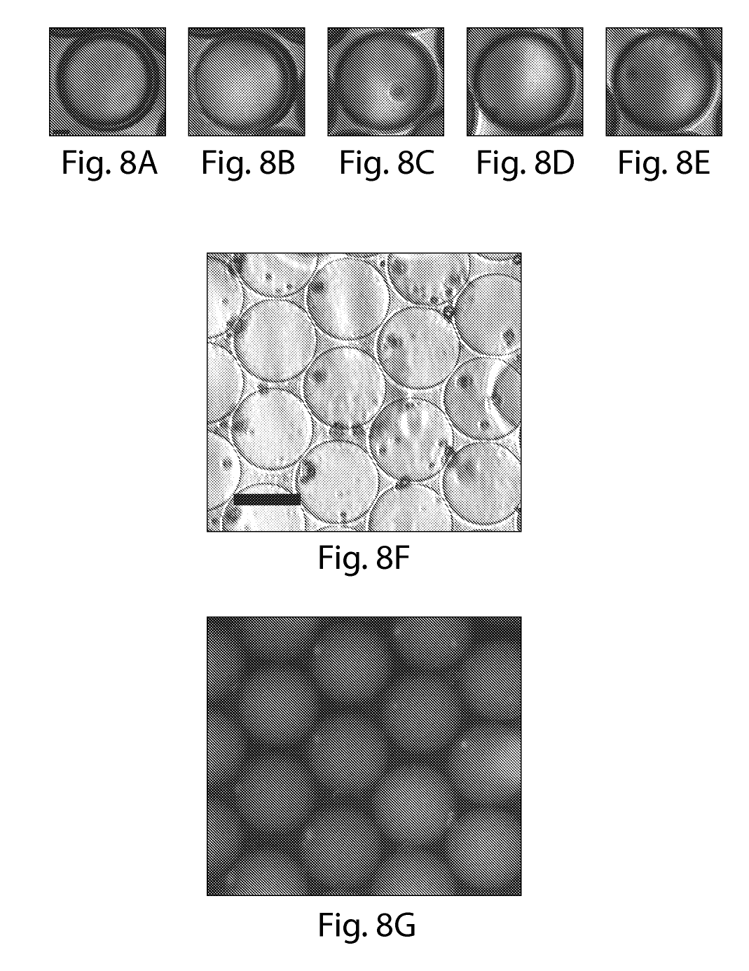

Any suitable method can be used to cause the fluidic droplet to release its contents. For example, a membrane material may be ruptured through a change in osmolarity, e.g., by increasing or decreasing the osmolarity. In some cases, the change in osmolarity may be fairly large, e.g., an increase of at least about 150%, at least about 200%, at least about 300%, etc., in osmolarity, or a decrease of at least about 50%, at least about 75%, or at least about 90% in osmolarity. As another example, a fluidic droplet containing a drug (e.g., within an inner fluidic droplet) may be chosen to dissolve, rupture, etc. under certain physiological conditions (e.g., pH, temperature, osmotic strength), allowing the drug to be selectively released. As yet another example, the fluidic droplet may be subjected to a chemical reaction, which disrupts the droplet and causes it to release its contents. In some cases, the chemical reaction may be externally initiated (e.g., upon exposure by the droplet to light, a chemical, a catalyst, etc.). As another example, a fluidic droplet may comprise a temperature-sensitive material. In one set of embodiments, the temperature-sensitive material changes phase upon heating or cooling, which may disrupt the material and allow release to occur. In another set of embodiments, the temperature-sensitive material shrinks upon heating or cooling. In some cases, shrinking of the material may cause the fluidic droplet to decease in size, causing release of its contents. An example of this process is shown in FIG. 7, which illustrates a vesicle subjected to osmotic shock.

As discussed, a vesicle can contain one or more species within the vesicle, e.g., within the inner fluid and/or within the membrane material. As an example, a cell can be suspended in a vesicle such as a liposome, a colloidosome, or a polymersome. The inner fluid may be, for example, an aqueous buffer solution. In a vesicle, the membrane material may be formed of a material capable of protecting the cell. The membrane may help retain, for example, moisture, and can be sized appropriately to maximize the lifetime of the cell within the vesicle. For instance, the vesicle may be sized to contain a specific volume, e.g., 10 nL, of inner fluid as well as a single cell or a select number of cells. Likewise, cells may be suspended in the bulk inner fluid so that, statistically, one cell will be included with each aliquot (e.g., 10 nL) of inner fluid when the inner fluid is used to form a vesicle.

One or more cells and/or one or more cell types can be contained in a vesicle. The inner fluid may be, for example, an aqueous buffer solution. The cell may be any cell or cell type. For example, the cell may be a bacterium or other single-cell organism, a plant cell, or an animal cell. If the cell is a single-cell organism, then the cell may be, for example, a protozoan, a trypanosome, an amoeba, a yeast cell, algae, etc. If the cell is an animal cell, the cell may be, for example, an invertebrate cell (e.g., a cell from a fruit fly), a fish cell (e.g., a zebrafish cell), an amphibian cell (e.g., a frog cell), a reptile cell, a bird cell, or a mammalian cell such as a primate cell, a bovine cell, a horse cell, a porcine cell, a goat cell, a dog cell, a cat cell, or a cell from a rodent such as a rat or a mouse. If the cell is from a multicellular organism, the cell may be from any part of the organism. For instance, if the cell is from an animal, the cell may be a cardiac cell, a fibroblast, a keratinocyte, a heptaocyte, a chondracyte, a neural cell, a osteocyte, a muscle cell, a blood cell, an endothelial cell, an immune cell (e.g., a T-cell, a B-cell, a macrophage, a neutrophil, a basophil, a mast cell, an eosinophil), a stem cell, etc. In some cases, the cell may be a genetically engineered cell. In certain embodiments, the cell may be a Chinese hamster ovarian (“CHO”) cell or a 3T3 cell.

Other examples of species that can be contained within a vesicle include, for example, other chemical, biochemical, or biological entities (e.g., dissolved or suspended in the fluid), particles, gases, molecules, pharmaceutical agents, drugs, DNA, RNA, proteins, fragrance, reactive agents, biocides, fungicides, preservatives, chemicals, or the like. Thus, the species may be any substance that can be contained in any portion of a vesicle and can be differentiated from the inner fluid. The species may be present in any portion of the vesicle.

As the polydispersity and size of the droplets can be narrowly controlled, emulsions or vesicles can be formed that include a specific number of species or particles. For instance, a single droplet may contain 1, 2, 3, 4, or more species. The emulsions or vesicles can be formed with low polydispersity so that greater than 90%, 95%, or 99% of those formed contain the same number of species. In certain instances, the invention provides for the production of vesicles consisting essentially of a substantially uniform number of entities of a species therein (i.e., molecules, cells, particles, etc.). For example, at least about 75%, at least about 80%, at least about 85%, at least about 90%, at least about 92%, at least about 94%, at least about 95%, at least about 96%, at least about 97%, at least about 98%, or at least about 99%, or more of a plurality or series of vesicle may each contain at least one entity, and/or may contain the same number of entities of a particular species. For instance, a substantial number of vesicles produced, e.g., as described above, may each contain 1 entity, 2 entities, 3 entities, 4 entities, 5 entities, 7 entities, 10 entities, 15 entities, 20 entities, 25 entities, 30 entities, 40 entities, 50 entities, 60 entities, 70 entities, 80 entities, 90 entities, 100 entities, etc., where the entities are molecules or macromolecules, cells, particles, etc. In some cases, the vesicles may each independently contain a range of entities, for example, less than 20 entities, less than 15 entities, less than 10 entities, less than 7 entities, less than 5 entities, or less than 3 entities in some cases.

In one set of embodiments, in a plurality of droplets of fluid, some of which contain a species of interest and some of which do not contain the species of interest, the droplets of fluid may be screened or sorted for those droplets of fluid containing the species, and in some cases, the droplets may be screened or sorted for those droplets of fluid containing a particular number or range of entities of the species of interest. Systems and methods for screening and/or sorting droplets are disclosed in, for example, U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference.

Thus, in some cases, a plurality or series of fluidic droplets or vesicles, some of which contain the species and some of which do not, may be enriched (or depleted) in the ratio of droplets that do contain the species, for example, by a factor of at least about 2, at least about 3, at least about 5, at least about 10, at least about 15, at least about 20, at least about 50, at least about 100, at least about 125, at least about 150, at least about 200, at least about 250, at least about 500, at least about 750, at least about 1000, at least about 2000, or at least about 5000 or more in some cases. In other cases, the enrichment (or depletion) may be in a ratio of at least about 10⁴, at least about 10⁵, at least about 10⁶, at least about 10⁷, at least about 10⁸, at least about 10⁹, at least about 10¹⁰, at least about 10¹¹, at least about 10¹², at least about 10¹³, at least about 10¹⁴, at least about 10¹⁵, or more. For example, a fluidic droplet or vesicle containing a particular species may be selected from a library of fluidic droplets or vesicles containing various species, where the library may have about 10⁵, about 10⁶, about 10⁷, about 10⁸, about 10⁹, about 10¹⁰, about 10¹¹, about 10¹², about 10¹³, about 10¹⁴, about 10¹⁵, or more items, for example, a DNA library, an RNA library, a protein library, a combinatorial chemistry library, etc.

As mentioned, in some aspects of the invention, vesicles such as those described herein are formed using multiple emulsions that are formed by flowing three (or more) fluids through a system of conduits. The system may be a microfluidic system. “Microfluidic,” as used herein, refers to a device, apparatus or system including at least one fluid channel having a cross-sectional dimension of less than about 1 millimeter (mm), and in some cases, a ratio of length to largest cross-sectional dimension of at least 3:1. One or more conduits of the system may be a capillary tube. In some cases, multiple conduits are provided, and in some embodiments, at least some are nested, as described herein. The conduits may be in the microfluidic size range and may have, for example, average inner diameters, or portions having an inner diameter, of less than about 1 millimeter, less than about 300 micrometers, less than about 100 micrometers, less than about 30 micrometers, less than about 10 micrometers, less than about 3 micrometers, or less than about 1 micrometer, thereby providing droplets having comparable average diameters. One or more of the conduits may (but not necessarily), in cross section, have a height that is substantially the same as a width at the same point. Conduits may include an orifice that may be smaller, larger, or the same size as the average diameter of the conduit. For example, conduit orifices may have diameters of less than about 1 mm, less than about 500 micrometers, less than about 300 micrometers, less than about 200 micrometers, less than about 100 micrometers, less than about 50 micrometers, less than about 30 micrometers, less than about 20 micrometers, less than about 10 micrometers, less than about 3 micrometers, etc. In cross-section, the conduits may be rectangular or substantially non-rectangular, such as circular or elliptical.

The conduit may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the conduit may be chosen such that fluid is able to freely flow through the article or substrate. The dimensions of the conduit may also be chosen, for example, to allow a certain volumetric or linear flowrate of fluid in the conduit. Of course, the number of conduits and the shape of the conduits can be varied by any method known to those of ordinary skill in the art.

The conduits of the present invention can also be disposed in or nested in another conduit, and multiple nestings are possible in some cases. In some embodiments, one conduit can be concentrically retained in another conduit and the two conduits are considered to be concentric. In other embodiments, however, one conduit may be off-center with respect to another, surrounding conduit. By using a concentric or nesting geometry, the inner and outer fluids, which are typically miscible, may avoid contact facilitating great flexibility in making multiple emulsions and in techniques for vesicle formation.

A flow pathway can exist in an inner conduit and a second flow pathway can be formed in a coaxial space between the external wall of the interior conduit and the internal wall of the exterior conduit, as discussed in detail below. The two conduits may be of different cross-sectional shapes in some cases. In one embodiment, a portion or portions of an interior conduit may be in contact with a portion or portions of an exterior conduit, while still maintaining a flow pathway in the coaxial space. Different conduits used within the same device may be made of similar or different materials. For example, all of the conduits within a specific device may be glass capillaries, or all of the conduits within a device may be formed of a polymer, for example, polydimethylsiloxane, as discussed below.

A geometry that provides coaxial flow can also provide hydrodynamic focusing of that flow, according to certain embodiments of the invention. Many parameters of the droplets, both inner droplets and middle layer droplets (outer droplets) can be controlled using hydrodynamic focusing. For instance, droplet diameter, outer droplet thickness and the total number of inner droplets per outer droplet can be controlled.

Multiple emulsion parameters can also be engineered by adjusting, for example, the system geometry, the flowrate of the inner fluid, the flowrate of the middle fluid and/or the flowrate of the outer fluid. By controlling these three flow rates independently, the number of internal droplets and the membrane thickness of the outer droplet (middle fluid) can be predicatively chosen.

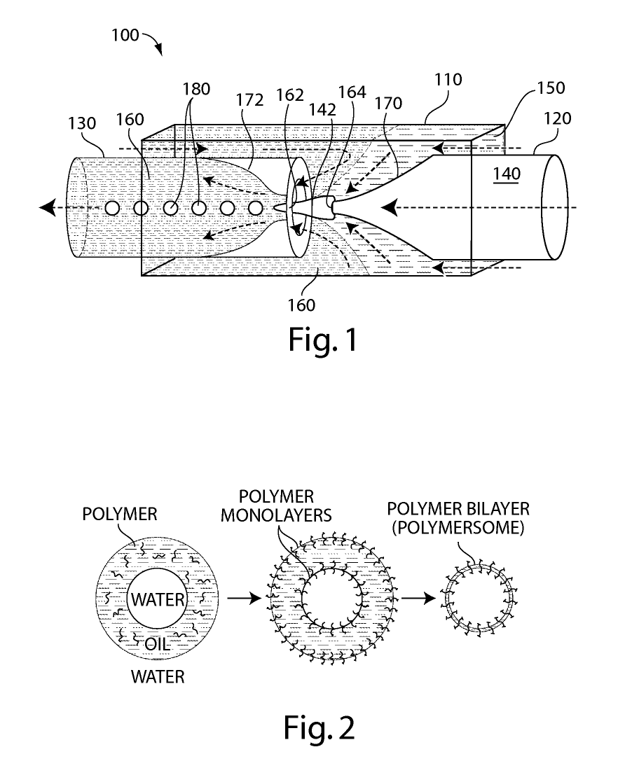

The schematic diagram illustrated in FIG. 1 shows one embodiment of the invention including a device 100 having an outer conduit 110, a first inner conduit (or injection tube) 120, and a second inner conduit (or collection tube) 130. An inner fluid 140 is shown flowing in a right to left direction and middle fluid 150 flows in a right to left direction in the space outside of injection tube 120 and within conduit 110. Outer fluid 160 flows in a left to right direction in the pathway provided between outer conduit 110 and collection tube 130. After outer fluid 160 contacts middle fluid 150, it changes direction and starts to flow in substantially the same direction as the inner fluid 140 and the middle fluid 150, right to left. Injection tube 120 includes an exit orifice 164 at the end of tapered portion 170. Collection tube 130 includes an entrance orifice 162, an internally tapered surface 172, and exit channel 168. Thus, the inner diameter of injection tube 120 decreases in a direction from right to left, as shown, and the inner diameter of collection tube 130 increases from the entrance orifice in a direction from right to left. These constrictions, or tapers, can provide geometries that aid in producing consistent multiple emulsions. The rate of constriction may be linear or non-linear.

As illustrated in FIG. 1, inner fluid 140 exiting from orifice 164 can be completely surrounded by middle fluid 150, as there is no portion of inner fluid 140 that contacts the inner surface of conduit 110 after its exit from injection tube 120. Thus, for a portion between exit orifice 164 to a point inside of collection tube 130 (to the left of entrance orifice 162), a stream of fluid 140 is concentrically surrounded by a stream of fluid 150. Additionally, middle fluid 150 may not come into contact with the surface of collection tube 130, at least until after the multiple emulsion has been formed, because it is concentrically surrounded by outer fluid 160 as it enters collection tube 130. Thus, from a point to the left of exit orifice 164 to a point inside of collection tube 130, a composite stream of three fluid streams is formed, including inner fluid 140 concentrically surrounded by a stream of middle fluid 150, which in turn is concentrically surrounded by a stream of outer fluid 160. The inner and middle fluids do not typically break into droplets until they are inside of collection tube 130 (to the left of entrance orifice 162). Under “dripping” conditions, the droplets are formed closer to the orifice, while under “jetting” conditions, the droplets are formed further downstream, i.e., to the left as shown in FIG. 1.

In addition, by controlling the geometry of the conduits and the flow of fluid through the conduits, the average diameters of the droplets may be controlled, and in some cases, controlled such that the average diameter of the droplets is less than about 1 mm, less than about 500 micrometers, less than about 200 micrometers, less than about 100 micrometers, less than about 75 micrometers, less than about 50 micrometers, less than about 25 micrometers, less than about 10 micrometers, or less than about 5 micrometers in some cases. Control of flow in such a fashion may be used to reduce the average diameters of the droplets in multiple emulsions.

The relative sizes of the inner fluid droplet and the middle fluid droplet can also be controlled, i.e., the ratio of the size of the inner and outer droplets can be predicatively controlled. For instance, inner fluid droplets may fill much of or only a small portion of the middle fluid (outer) droplet. Inner fluid droplets may fill less than about 90%, less than about 80%, less than about 70%, less than about 60%, less than about 50%, less than about 30%, less than about 20%, or less than about 10% of the volume of the outer droplet. Alternatively, the inner fluid droplet may form greater than about 10%, about 20%, about 30%, about 40%, about 50%, about 60%, about 70%, about 90%, about 95%, or about 99% of the volume of the outer droplet. In some cases, the outer droplet can be considered a fluid membrane when it contains an inner droplet, as some or most of the outer droplet volume may be filled by the inner droplet. The ratio of the middle fluid membrane thickness to the middle fluid droplet radius can be equal to or less than, e.g., about 5%, about 4%, about 3%, or about 2%. This can allow, in some embodiments, for the formation of multiple emulsions with only a very thin layer of material separating, and thus stabilizing, two miscible fluids. The middle material can also be thickened to greater than or equal to, e.g., about 10%, about 20%, about 30%, about 40%, or about 50% of the middle fluid droplet radius.

In some cases, such as when droplets of middle fluid 150 (outer droplets) are formed at the same rate as are droplets of inner fluid 140, then there is a one-to-one correspondence between inner fluid and middle fluid droplets, and each droplet of inner fluid is surrounded by a droplet of middle fluid, and each droplet of middle fluid contains a single inner droplet of inner fluid. The term “outer droplet,” as used herein, typically means a fluid droplet containing an inner fluid droplet that comprises a different fluid. In many embodiments that use three fluids for multiple emulsion production, the outer droplet is formed from a middle fluid and not from the outer fluid as the term may imply. It should be noted that the above-described figure is by way of example only, and other devices are also contemplated within the instant invention. For example, the device in FIG. 1 may be modified to include additional concentric tubes, for example, to produce more highly nested droplets.

The rate of production of multiple emulsion droplets may be determined by the droplet formation frequency, which under many conditions can vary between approximately 100 Hz and 5,000 Hz. In some cases, the rate of droplet production may be at least about 200 Hz, at least about 300 Hz, at least about 500 Hz, at least about 750 Hz, at least about 1,000 Hz, at least about 2,000 Hz, at least about 3,000 Hz, at least about 4,000 Hz, or at least about 5,000 Hz.

Production of large quantities of vesicles can be facilitated by the parallel use of multiple devices in some instances. In some cases, relatively large numbers of devices may be used in parallel, for example at least about 10 devices, at least about 30 devices, at least about 50 devices, at least about 75 devices, at least about 100 devices, at least about 200 devices, at least about 300 devices, at least about 500 devices, at least about 750 devices, or at least about 1,000 devices or more may be operated in parallel. The devices may comprise different conduits (e.g., concentric conduits), orifices, microfluidics, etc. In some cases, an array of such devices may be formed by stacking the devices horizontally and/or vertically. The devices may be commonly controlled, or separately controlled, and can be provided with common or separate sources of inner, middle, and outer fluids, depending on the application.

Production of large quantities of emulsions can be facilitated by the parallel use of multiple devices such as those described herein, in some instances. In some cases, relatively large numbers of devices may be used in parallel, for example at least about 10 devices, at least about 30 devices, at least about 50 devices, at least about 75 devices, at least about 100 devices, at least about 200 devices, at least about 300 devices, at least about 500 devices, at least about 750 devices, or at least about 1,000 devices or more may be operated in parallel. The devices may comprise different conduits (e.g., concentric conduits), orifices, microfluidics, etc. In some cases, an array of such devices may be formed by stacking the devices horizontally and/or vertically. The devices may be commonly controlled, or separately controlled, and can be provided with common or separate sources of various fluids, depending on the application.

Accordingly, a variety of materials and methods, according to certain aspects of the invention, can be used to form any of the above-described components of the systems and devices of the invention, for example, microfluidic channels for forming various vesicles as described above. In some cases, the various materials selected lend themselves to various methods. For example, various components of the invention can be formed from solid materials, in which the channels can be formed via micromachining, film deposition processes such as spin coating and chemical vapor deposition, laser fabrication, photolithographic techniques, etching methods including wet chemical or plasma processes, and the like. See, for example, Scientific American, 248:44-55, 1983 (Angell, et al). In one embodiment, at least a portion of the fluidic system is formed of silicon by etching features in a silicon chip. Technologies for precise and efficient fabrication of various fluidic systems and devices of the invention from silicon are known. In another embodiment, various components of the systems and devices of the invention can be formed of a polymer, for example, an elastomeric polymer such as polydimethylsiloxane (“PDMS”), polytetrafluoroethylene (“PTFE” or Teflon®), or the like.

Different components can be fabricated of different materials. For example, a base portion including a bottom wall and side walls can be fabricated from an opaque material such as silicon or PDMS, and a top portion can be fabricated from a transparent or at least partially transparent material, such as glass or a transparent polymer, for observation and/or control of the fluidic process. Components can be coated so as to expose a desired chemical functionality to fluids that contact interior channel walls, where the base supporting material does not have a precise, desired functionality. For example, components can be fabricated as illustrated, with interior channel walls coated with another material. Material used to fabricate various components of the systems and devices of the invention, e.g., materials used to coat interior walls of fluid channels, may desirably be selected from among those materials that will not adversely affect or be affected by fluid flowing through the fluidic system, e.g., material(s) that is chemically inert in the presence of fluids to be used within the device.

In one embodiment, various components of the invention are fabricated from polymeric and/or flexible and/or elastomeric materials, and can be conveniently formed of a hardenable fluid, facilitating fabrication via molding (e.g. replica molding, injection molding, cast molding, etc.). The hardenable fluid can be essentially any fluid that can be induced to solidify, or that spontaneously solidifies, into a solid capable of containing and/or transporting fluids contemplated for use in and with the fluidic network. In one embodiment, the hardenable fluid comprises a polymeric liquid or a liquid polymeric precursor (i.e. a “prepolymer”). Suitable polymeric liquids can include, for example, thermoplastic polymers, thermoset polymers, or mixture of such polymers heated above their melting point. As another example, a suitable polymeric liquid may include a solution of one or more polymers in a suitable solvent, which solution forms a solid polymeric material upon removal of the solvent, for example, by evaporation. Such polymeric materials, which can be solidified from, for example, a melt state or by solvent evaporation, are well known to those of ordinary skill in the art. A variety of polymeric materials, many of which are elastomeric, are suitable, and are also suitable for forming molds or mold masters, for embodiments where one or both of the mold masters is composed of an elastomeric material. A non-limiting list of examples of such polymers includes polymers of the general classes of silicone polymers, epoxy polymers, and acrylate polymers. Epoxy polymers are characterized by the presence of a three-membered cyclic ether group commonly referred to as an epoxy group, 1,2-epoxide, or oxirane. For example, diglycidyl ethers of bisphenol A can be used, in addition to compounds based on aromatic amine, triazine, and cycloaliphatic backbones. Another example includes the well-known Novolac polymers. Non-limiting examples of silicone elastomers suitable for use according to the invention include those formed from precursors including the chlorosilanes such as methylchlorosilanes, ethylchlorosilanes, phenylchlorosilanes, etc.

Silicone polymers are preferred in one set of embodiments, for example, the silicone elastomer polydimethylsiloxane. Non-limiting examples of PDMS polymers include those sold under the trademark Sylgard by Dow Chemical Co., Midland, Mich., and particularly Sylgard 182, Sylgard 184, and Sylgard 186. Silicone polymers including PDMS have several beneficial properties simplifying fabrication of the microfluidic structures of the invention. For instance, such materials are inexpensive, readily available, and can be solidified from a prepolymeric liquid via curing with heat. For example, PDMSs are typically curable by exposure of the prepolymeric liquid to temperatures of about, for example, about 65° C. to about 75° C. for exposure times of, for example, about an hour. Also, silicone polymers, such as PDMS, can be elastomeric, and thus may be useful for forming very small features with relatively high aspect ratios, necessary in certain embodiments of the invention. Flexible (e.g., elastomeric) molds or masters can be advantageous in this regard.

One advantage of forming structures such as microfluidic structures of the invention from silicone polymers, such as PDMS, is the ability of such polymers to be oxidized, for example by exposure to an oxygen-containing plasma such as an air plasma, so that the oxidized structures contain, at their surface, chemical groups capable of cross-linking to other oxidized silicone polymer surfaces or to the oxidized surfaces of a variety of other polymeric and non-polymeric materials. Thus, components can be fabricated and then oxidized and essentially irreversibly sealed to other silicone polymer surfaces, or to the surfaces of other substrates reactive with the oxidized silicone polymer surfaces, without the need for separate adhesives or other sealing means. In most cases, sealing can be completed simply by contacting an oxidized silicone surface to another surface without the need to apply auxiliary pressure to form the seal. That is, the pre-oxidized silicone surface acts as a contact adhesive against suitable mating surfaces. Specifically, in addition to being irreversibly sealable to itself, oxidized silicone such as oxidized PDMS can also be sealed irreversibly to a range of oxidized materials other than itself including, for example, glass, silicon, silicon oxide, quartz, silicon nitride, polyethylene, polystyrene, glassy carbon, and epoxy polymers, which have been oxidized in a similar fashion to the PDMS surface (for example, via exposure to an oxygen-containing plasma). Oxidation and sealing methods useful in the context of the present invention, as well as overall molding techniques, are described in the art, for example, in an article entitled “Rapid Prototyping of Microfluidic Systems and Polydimethylsiloxane,” Anal. Chem., 70:474-480, 1998 (Duffy, et al.), incorporated herein by reference.

In some embodiments, certain microfluidic structures of the invention (or interior, fluid-contacting surfaces) may be formed from certain oxidized silicone polymers. Such surfaces may be more hydrophilic than the surface of an elastomeric polymer. Such hydrophilic channel surfaces can thus be more easily filled and wetted with aqueous solutions.

In one embodiment, a bottom wall of a microfluidic device of the invention is formed of a material different from one or more side walls or a top wall, or other components. For example, the interior surface of a bottom wall can comprise the surface of a silicon wafer or microchip, or other substrate. Other components can, as described above, be sealed to such alternative substrates. Where it is desired to seal a component comprising a silicone polymer (e.g. PDMS) to a substrate (bottom wall) of different material, the substrate may be selected from the group of materials to which oxidized silicone polymer is able to irreversibly seal (e.g., glass, silicon, silicon oxide, quartz, silicon nitride, polyethylene, polystyrene, epoxy polymers, and glassy carbon surfaces which have been oxidized). Alternatively, other sealing techniques can be used, as would be apparent to those of ordinary skill in the art, including, but not limited to, the use of separate adhesives, thermal bonding, solvent bonding, ultrasonic welding, etc.

The following applications are each incorporated herein by reference: U.S. patent application Ser. No. 11/885,306, filed Aug. 29, 2007, entitled “Method and Apparatus for Forming Multiple Emulsions,” by Weitz, et al.; U.S. patent application Ser. No. 12/058,628, filed Mar. 28, 2008, entitled “Emulsions and Techniques for Formation,” by Chu, et al.; U.S. patent application Ser. No. 11/246,911, filed Oct. 7, 2005, entitled “Formation and Control of Fluidic Species,” by Link, et al., published as U.S. Patent Application Publication No. 2006/0163385 on Jul. 27, 2006; U.S. patent application Ser. No. 11/024,228, filed Dec. 28, 2004, entitled “Method and Apparatus for Fluid Dispersion,” by Stone, et al., published as U.S. Patent Application Publication No. 2005/0172476 on Aug. 11, 2005; and U.S. patent application Ser. No. 11/360,845, filed Feb. 23, 2006, entitled “Electronic Control of Fluidic Species,” by Link, et al., published as U.S. Patent Application Publication No. 2007/000344 on Jan. 4, 2007. Also incorporated herein by reference is U.S. Provisional Patent Application Ser. No. 61/059,163, filed Jun. 5, 2008, entitled “Polymersomes, Liposomes, and other Species Associated with Fluidic Droplets,” by Shum, et al.

The following examples are intended to illustrate certain embodiments of the present invention, but do not exemplify the full scope of the invention.

### Example 1

The encapsulation of drugs, flavors, colorings, fragrance and other active agents is of increasing importance to the pharmaceutical, food, beverage, and cosmetic industries. Ideal encapsulating structures should capture the actives as efficiently as possible and should be easily triggered to release the actives. One class of suitable structures includes vesicles, which are microscopic compartments enclosed by a thin membrane often self-assembled from amphiphilic molecules. Due to the hydrophobicity of the membrane, active materials with large sizes cannot readily pass through the vesicle wall; however, small molecules such as water can penetrate the vesicles. Therefore, depending on the osmotic pressure difference between the aqueous core and the surrounding environment, vesicles can be inflated or deflated by varying the water content. The thin membrane that makes up the vesicle wall is often mechanically weak and breaks beyond a certain pressure difference, releasing the actives. This provides a simple mechanism for triggered release.

This example describes a microfluidic approach for fabricating monodisperse biocompatible poly(ethylene glycol)-poly(lactic acid) (PEG-PLA) polymersomes that selectively encapsulate hydrophilic solutes with high encapsulation efficiency. This example uses monodisperse double emulsion as templates to direct the assembly of PEG-b-PLA during solvent evaporation. The polymersomes prepared encapsulate a fluorescent hydrophilic solute, which can be released by application of a large osmotic pressure difference. This example also shows that this technique can be used with diblock copolymers with different molecular weight ratio of the hydrophilic and the hydrophobic blocks. Depending on the ratio, the wetting angle of the polymer containing solvent phase on the polymersomes changes in the emulsion-to-polymersomes transition. The property of the polymersome wall can also be tuned by changing the block ratio. Thus, these techniques allow the fabrication of PEG-b-PLA polymersomes with excellent encapsulation efficiency, high levels of actives loading, or tunable wall properties.

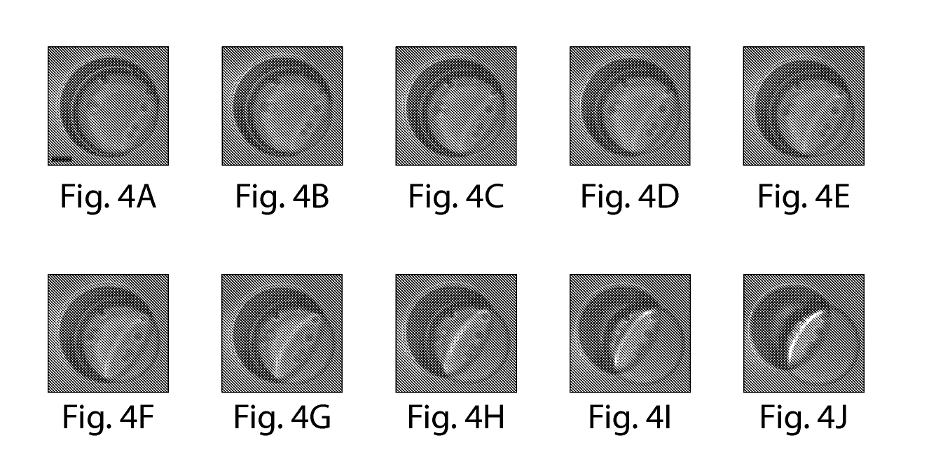

Formation of block copolymer-stabilized double emulsions. Monodisperse W/O/W double emulsions stabilized by a diblock polymer of PEG(5000)-b-PLA(5000) were prepared in glass microcapillary devices, as shown schematically in FIG. 3. In this example, the outer phase 205 was substantially immiscible with the middle phase 215, which was in turn substantially immiscible with the inner phase 225. However, the inner phase may be miscible with the outer phase. Both the injection tube 210 and the collection tube 220 were tapered from glass capillary tubes with an outer diameter of about 1,000 micrometers and an inner diameter of about 580 micrometers. Typical inner diameters after tapering ranged from about 10 micrometers to about 50 micrometers for the injection tube and from about 40 micrometers to about 100 micrometers for the collection tube. The fluorescence dye-containing inner drops were formed in the dripping regime from the small injection tube in a coflow geometry while the middle oil stream containing the inner drops was flow-focused by the outer continuous phase and breaks up into double emulsion drops. Since the inner phase was in contact with an immiscible middle oil phase, fluorescence dyes were retained in the inner phase without leakage to the outer continuous phase during the emulsion fabrication. The middle phase included PEG(5000)-b-PLA(5000) dissolved in a mixture of toluene and chloroform in a volume ratio of 2:1. The appropriate solvent should be highly volatile and dissolve the diblock copolymer well. While the PEG(5000)-b-PLA(5000) had a high solubility in chloroform, double emulsions with chloroform alone as the middle oil layer had a higher density than the aqueous continuous phase. The double emulsion drops therefore sank to the bottom of the container. Toluene has a lower density than the continuous phase, but it did not dissolve the copolymer as well. The mixture of toluene and chloroform in a 2:1 volume ratio was found to provide a reasonable combination of the properties.

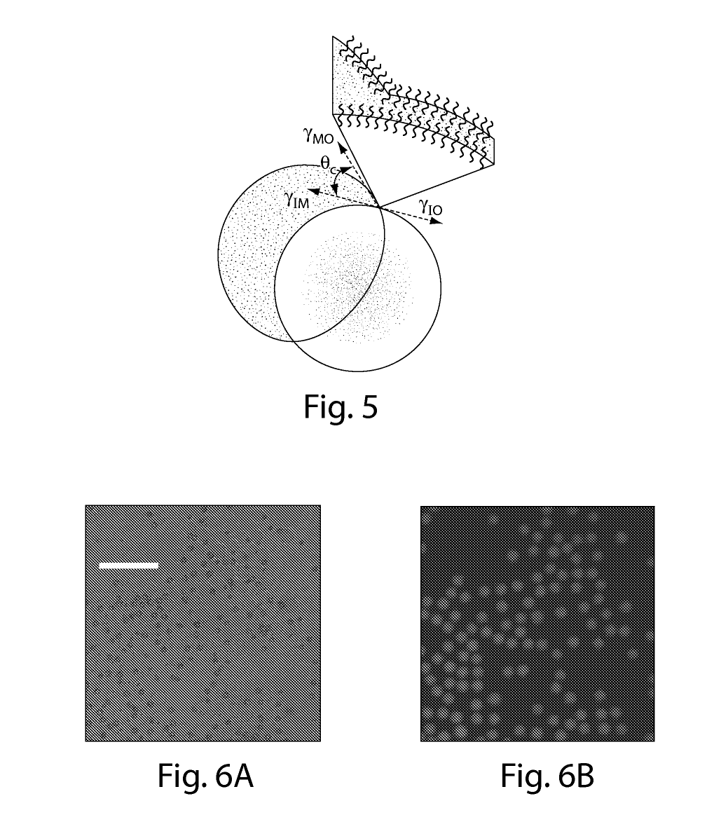

Transition from double emulsions to polymersomes. Double emulsion drops stabilized by the PEG(5000)-b-PLA(5000) copolymers typically went through various stages of dewetting transition, as shown in FIG. 4. This figure shows bright-field microscope images of a double emulsion drop undergoing dewetting transition. The double emulsion drop included an aqueous drop surrounded by a shell of 10 mg·mL⁻¹ PEG(5000)-b-PLA(5000) diblock copolymer dissolved in a toluene/chloroform mixture (2:1 by volume). At the end of the transition (FIG. 4J), the drop adopted an acorn-like structure with the organic solvent drop on the left and the aqueous drop on the right. Successive images were taken at intervals of 910 ms. Scale bar is 10 micrometers.

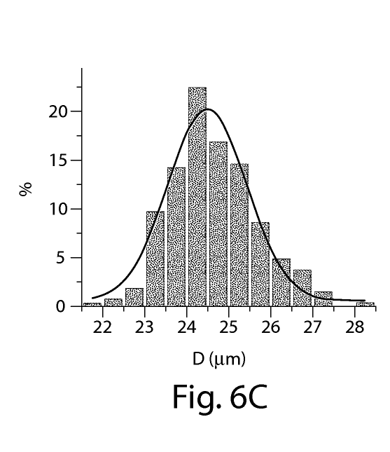

The organic solvent layer, which initially wets the entire inner drop, dewetted from the inner drop, resulting in an acorn-like structure. The contact angle, θ_{c}, at the three phase contact point was 56°, as schematically illustrated in FIG. 5, showing partial wetting of the organic phase on a thin layer of block copolymer. The acorn-like equilibrium structure was predicted from an analysis of the three interfacial tensions between various different pairs of three immiscible liquids. The final morphology of a core-shell system appeared to be determined by the relative surface energies. If the interface between the core and the external phase had a larger surface energy compared with that between the core and the shell, the shell completely wetted the core, forming a stable core-shell structure. If the relative surface energy between the core and the shell phase was very high, the core and the shell separated from each other to avoid wetting. In the case of comparable surface energies, partial wetting between the core and the shell occurred, leading to formation of acorn-like structures. Each of the morphologies was observed experimentally in a three-phase system of oil, water and polymer. The PEG(5000)-b-PLA(5000) copolymer acted as a surfactant and migrates to the two interfaces. The formation of acorn-like structures suggested that the surface energy of the copolymer-oil interface was comparable to that of the copolymer bilayer. From a force balance at the three phase contact point shown in FIG. 5, for this partial wetting to occur, there must be a negative spread coefficient, S, such that:

S=γ_{IO}−γ_{IM}−γ_{MO},

where γ_{IO}, γ_{IM} and γ_{MO} are the surface tensions of the inner-outer, the inner-middle and the middle-outer interfaces respectively. In these experiments, the measured value of the spreading coefficient was −2.1 mN/m. Associated with S was an attractive adhesion energy between the inner and outer phases, and the driving force for the attraction has been shown to arise from depletion effects.

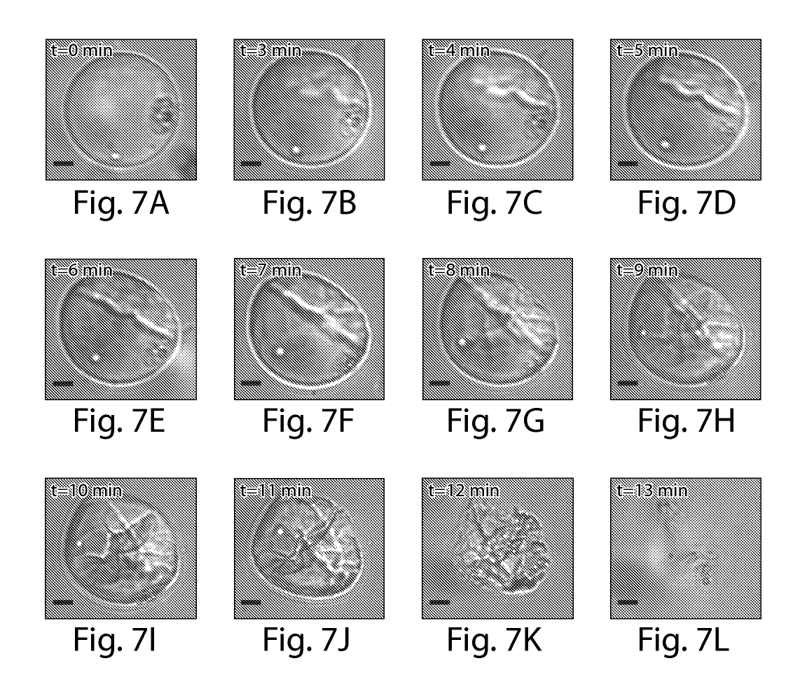

Monodisperse polymersomes for encapsulation. One bulb of the acorn-like dewetted drop included a volatile organic solvent, which continued to evaporate after the dewetting transition. The evaporation rate can be adjusted to ensure that the double emulsion remains stable throughout the evaporation process. After evaporation of the organic solvent for about a day, the excess diblock copolymer formed an aggregate on the side where the organic solvent drop attaches (FIG. 6A). This figure shows a bright-field microscope image of the PEG(5000)-b-PLA(5000) polymersomes formed after dewetting transition and solvent evaporation. The excess diblock copolymer contained in the dewetted organic solvent drop appeared to form the aggregates, which were attached to the polymersomes. Occasionally, the aggregates were detached from the polymersomes, as shown in the red box. Scale bar is 100 micrometers.

The size of the aggregates attached to the polymersomes may also be controlled by varying the amount of excess diblock copolymer in the organic solvent layer. Occasionally, the oil drop, as it is drying, can break off the polymersome, carrying the excess diblock copolymer and leaving behind a homogeneous polymersome (see box in FIG. 6A). Thus, in some cases, homogeneous polymersomes may be obtained with gentle stirring. This offers a simple and effective route to obtain spherical homogeneous polymersomes if the gentile stirring is performed in a controlled fashion.

Due to the small difference between the refractive indices of the inner and the outer phases, the polymersomes could barely be seen in bright field microscopy. In fluorescence microscopy, however, the polymersomes could be clearly seen as bright green spots, as shown in FIG. 6B, which is a fluorescence microscope image of the same area as in FIG. 6A. The fluorescent HPTS solutes were well-encapsulated inside the polymersomes without leakage to the continuous phase. The large contrast in fluorescence intensity between the inner drop and the outer continuous phase demonstrates the encapsulation efficiency of the fabrication process. Not only is the FITC-Dextran, with an average molecular weight of 4000 Da, well encapsulated, but remarkably, the fluorescent HPTS dye, with a very small molecular weight of less than 600 Da, also stayed encapsulated inside the polymersomes. This highlights the low membrane permeability to small hydrophilic solutes. After going through the processes of dewetting and solvent evaporation, the polymersomes still showed a low polydispersity of only 4% or lower, as determined by image analysis. In particular, FIG. 6C shows the size distribution of the PEG(5000)-b-PLA(5000) polymersomes. The polydispersity of polymersomes is 4.0%. The experimental data is fitted with a Gaussian distribution.

In the polymersome fabrication process, the osmolalities of the inner phase and the outer phase were balanced to maintain the polymersome size. In some initial experimental runs where sodium chloride salt is not added to balance the osmolality with the outer solution, the polymersomes shrank considerably after dewetting. Although the membrane was generally impermeable to the small HPTS salts, water molecules could diffuse in and out of the polymersomes. The osmotic pressure, πₒₛₘ, was related to the concentration of solutes:

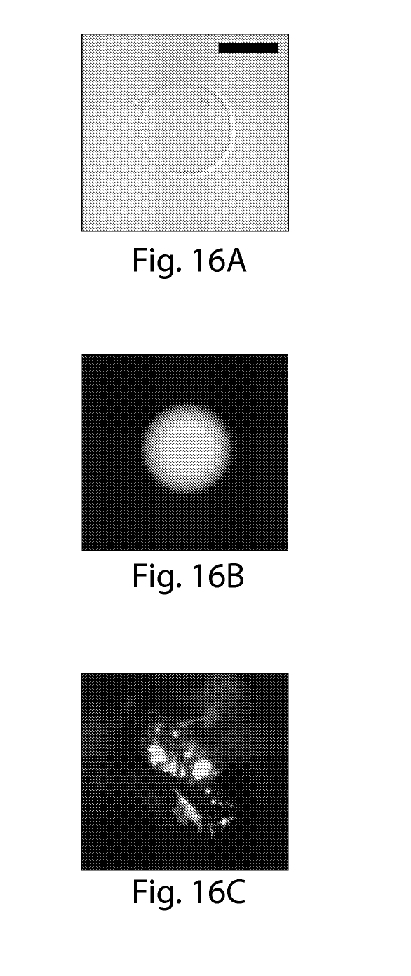

ρₒₛₘ=cRT,

where c is the molar concentration of the solutes, R is the gas constant and T is the temperature. Due to osmotic pressure difference, water diffuses from regions with a low salt concentration to regions with a higher concentration. Osmotic pressure could therefore be used to tune the sizes of the polymersomes. If the osmotic pressure change was sudden and large, the resulting shock may break the polymersomes in some cases (see FIG. 15). The kinetics of the response of the polymersomes following a large osmotic shock was too fast to visualize; in these experiments, the process for visualization was slowed down by gradually increasing PVA concentration through water evaporation as is shown in FIG. 7, which shows bright-field microscope images showing the shrinkage and breakage of a PEG(5000)-b-PLA(5000) polymersome after an osmotic shock. As a result of water expulsion from its inside, the polymersome shrank and wrinkled. By tuning the wall properties such as its crystallinity, the polymersome wall could break. Scale bar is 10 micrometers.

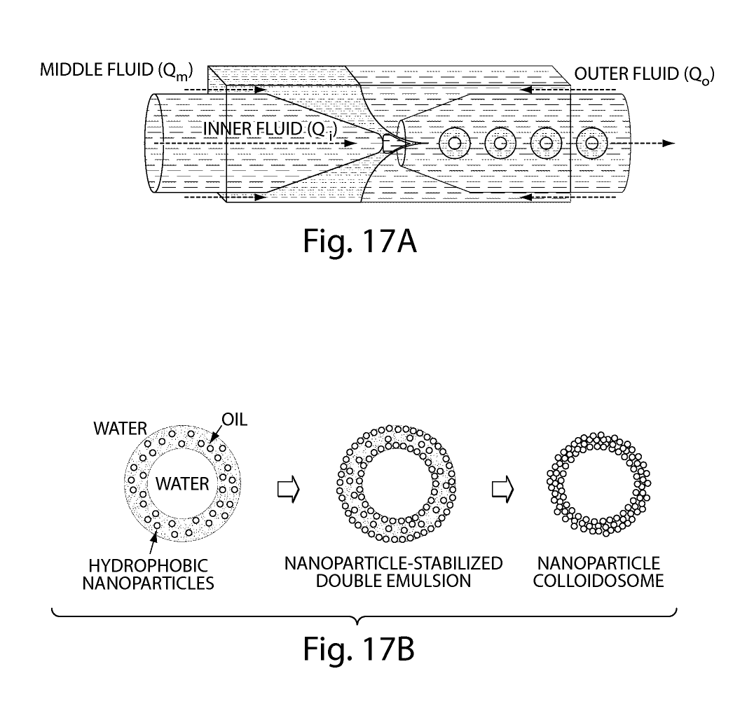

Initially, the polymersomes were suspended in a 10 wt % PVA solution, which was left to evaporate in air on a glass slide. As the water evaporated, the PVA concentration became higher and higher and so water was squeezed out from the inside of the polymersome. As a result, the polymersome becomes smaller, and its wall buckled, as shown in FIG. 16. When subjected to a sufficiently high osmotic shock, the polymersome wall can break (see FIG. 16). This provides a simple trigger for the release of the encapsulated fluorescent. Thus, by tuning the properties of the polymersome wall, it is possible to adjust the level of osmotic shock required to break the polymersomes. Alternatively, release can be triggered by diluting the continuous phase and thus reducing its osmotic pressure.

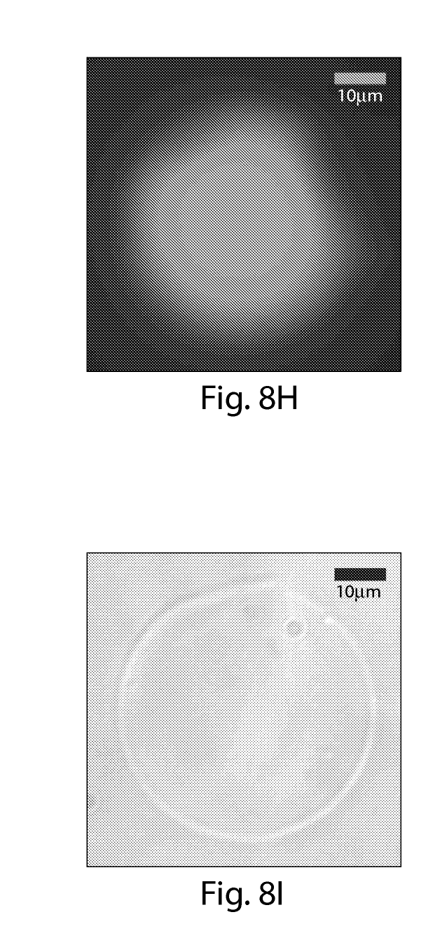

Copolymers with different block ratios. The same technique was also applied to diblock copolymers of different block ratios. With a PLA-rich diblock copolymer of PEG(1000)-b-PLA(5000), double emulsions collected did not form the acorn-like structures observed in the case of PEG(5000)-b-PLA(5000) (FIG. 8A-8E). As the organic solvent evaporates, the middle solvent phase gets thinner and thinner. Eventually, after most of the organic solvent was evaporated, dewetting of the middle phase occurred and aggregates were seen attached to the final capsules, similar to those attached to the PEG(5000)-b-PLA(5000) polymersomes (FIG. 8F). FIG. 8F shows a bright-field microscope image of a dried capsule formed from the PEG(1000)-b-PLA(5000) diblock copolymer. The arrows indicate aggregates of excess diblock copolymer. Scale bar is 50 micrometers. However, the contact angle of the middle phase at the three phase contact point was much smaller (about 17°). The spreading coefficient associated with it was −0.4 mN/m. This suggested that the organic solvent with the PLA-rich diblock copolymer wetted the inner drop more than that with PEG(5000)-b-PLA(5000). FIGS. 8A-8E show a series of bright-field microscope image following the evaporation of the organic solvent shell of a double emulsion drop. The double emulsion drop included an aqueous drop surrounded by a shell of 10 mg·mL⁻¹ PEG(1000)-b-PLA(5000) diblock copolymer dissolved in a toluene/chloroform mixture (2:1 by volume). The shell gets thinner and thinner as the toluene/chloroform mixture evaporates. Scale bar is 10 micrometers. The images were taken at intervals of 1 hr.

Like the PEG(5000)-b-PLA(5000) polymersomes, these capsules showed encapsulation of both the FITC-Dextran (FIG. 8H) and the low molecular weight HPTS (FIG. 8G), which could be released by application of an osmotic pressure shock. This figure shows a fluorescence microscope image of the same area as in FIG. 8F. As in the case of the PEG(5000)-b-PLA(5000), the fluorescent HPTS solutes were well-encapsulated inside, without leakage to the continuous phase.

It was also demonstrated that FITC-Dextran was released from the PEG(1000)-b-PLA(5000) polymersomes by diluting the continuous phase with water. Before dilution, FITC-Dextran was encapsulated inside the polymersomes, as shown by the green fluorescent compartment in FIG. 8H, which shows a fluorescence microscope image of a PEG(1000)-b-PLA(5000) polymersome encapsulating the green FITC-Dextran in a 1M Trizma buffer solution (pH 7.2). The polymersome was slightly deflated initially when the salt concentration in the continuous phase is higher due to water evaporation. After dilution with water, the green fluorescence of the polymersome disappeared even though the polymersome was still observed in bright field, as shown in FIG. 8I. This figure is a bright-field microscope image of a PEG(1000)-b-PLA(5000) polymersome after dilution of the continuous phase by about five times with deionized water. Even though the polymersome is visible in bright field, no fluorescence can be observed in fluorescence microscopy, indicating that the FITC-Dextran has been released after dilution of the continuous phase with water. To ensure that this is not an artifact due to photo-bleaching of the FITC-Dextran, the fluorescent shutter remained closed at all times except when the polymersomes are imaged about ten minutes after dilution with water. The contrast in fluorescence intensity appeared to be too low for the polymersomes to be observed with fluorescence microscopy after the osmotic shock. To better visualize the polymersome, bright-field microscopy was used. These images suggested that the polymersome remains intact after the osmotic shock; nevertheless, the FITC-Dextran was released when the osmotic pressure outside the polymersomes was decreased. However, the FITC-Dextran may be released from the polymersomes through cracks or pores that are too small to be observed.

The versatility of this technique to diblock copolymers of different PEG/PLA ratios allows customization of polymersomes for different technological applications. By changing the PLA/PEG ratio, blends of PLA and PEG exhibit different properties such as morphology, crystallinity, mechanical properties, or degradation properties.

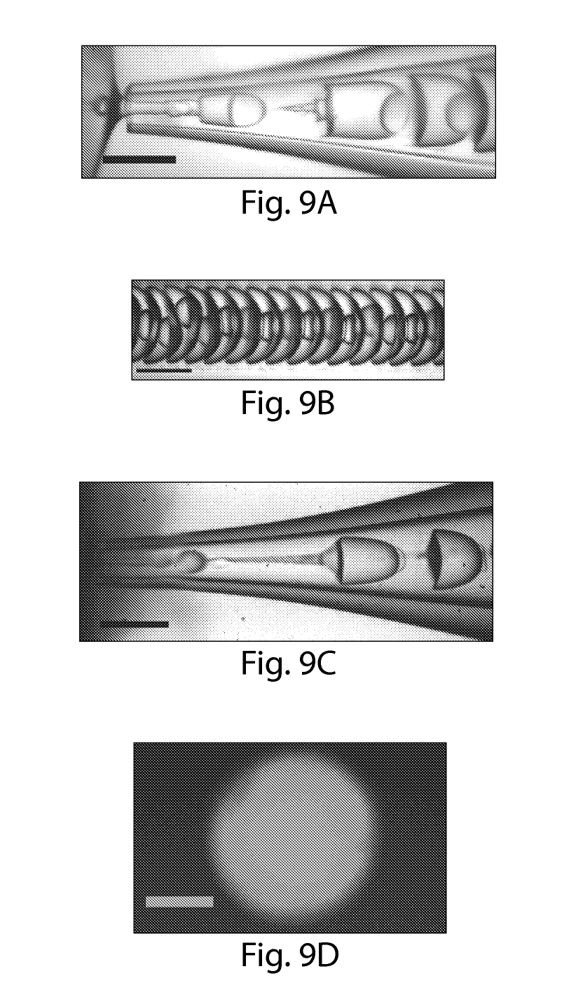

The diblock copolymer, PEG(5000)-b-PLA(1000), appeared to be surface active and lowers the interfacial tension significantly, as suggested by the highly non-spherical shape of the droplets in FIG. 9; an interface with a higher interfacial tension would otherwise relax to the surface-minimizing spherical shape quickly. In FIG. 9A, the middle phase that forms the shell included 5 mg/mL PEG(5000)-b-PLA(1000) and 2 mg/mL PLA homopolymer dissolved in pure toluene. However, using this diblock copolymer, double emulsions did not appear to be stable until additional PLA homopolymer was added to the middle phase; then double emulsion drops were generated (FIG. 9A) and the inner drops remained stable inside the middle drops (FIG. 9B). Without the PLA homopolymer, the inner drops broke through the middle phase almost immediately after generation of double emulsion drops, as shown in FIG. 9C; as a result, only a simple emulsion of the middle phase was collected. This suggested that addition of the PLA homopolymer may increase the double emulsion stability. The resulting polymersomes demonstrated encapsulation behavior (FIG. 9D, which shows a polymersome encapsulating fluorescent solutes obtained from the double emulsions shown in FIGS. 9A and 9B after solvent evaporation.). The scale bar is 300 micrometers for FIGS. 9A-9C and 30 micrometers for FIG. 9D.

The idea of incorporating polymersomes with homopolymer has also been demonstrated using common polymersome formation techniques such as rehydration. These technique allow the fabrication of more uniform polymersomes with a simple and efficient way of actives encapsulation. By incorporating different homopolymers to modify the properties and morphology, these techniques can be applied to engineer uniform macromolecular structures with controllable properties.

Details regarding the above experiments follow. Preparation of monodisperse double emulsions. Water-in-oil-in-water (W/O/W) double emulsion drops were produced using glass microcapillary devices. The inner phase included 0.6 wt % fluorescein isothiocyanate-dextran (FITC-Dextran; M_{w}: 4000) or 2.67 mM 8-hydroxypyrene-1,3,6-trisulfonic acid trisodium salt (HPTS) in water. Sodium chloride was added in some experiments to achieve the same osmalality with the outer phase. The osmalility of the solutions are measured with a microosmometer (Advanced Instruments, Inc., Model 3300). Unless otherwise noted, the middle hydrophobic phase was 5-10 mg·mL⁻¹ diblock polymer in an organic solvent of toluene and chloroform mixed in 2-to-1 volume ratio. Experiments were conducted with biodegradable copolymers of polylactic acid (PLA) and polyethylene glycol (PEG) with different block molecular weight ratios: PEG-b-PLA (1000 g·mol⁻¹/5000 g·mol⁻¹), (5000 g·mol⁻¹/5000 g·mol⁻¹) and (5000 g·mol⁻¹/1000 g·mol⁻¹) as well as a homopolymer of poly(dl-lactic acid) (PLA; M_{w}: 6000-16000 g·mol⁻¹). The outer phase was a 10 wt % poly(vinyl alcohol) aqueous solution (PVA; M_{w}: 13000-23000 g·mol⁻¹, 87-89% hydrolyzed). The diblock polymers stabilized the inner drops against coalescence with the exterior aqueous phase, while PVA prevented coalescence of the oil drops. The diblock copolymers and the homopolymer were obtained from Polysciences, Inc. while all other chemicals were obtained from Aldrich. Water with a resistivity of 18.2 Megohm cm⁻¹ was acquired from a Millipore Milli-Q system.

Formation of polymersomes. Monodisperse W/O/W double emulsions were prepared in glass microcapillary devices, as shown schematically in FIG. 3. The inner drops formed at the tip of the small injection tube in a coflow geometry while the middle oil stream, containing the inner drops, broke up into drops in the collection tube. The outer radii, Rₒ, of the double emulsions varied from 15 to 40 micrometers, while the inner radii, Rᵢ, varied from 12 to 30 micrometers. These values were controlled by the size of the capillaries used and the flow rates of the different phases. Typically, the volume of the middle phase was 1 to 10 times the volume of the inner phase. The formation of polymersomes by evaporation of the solvent was monitored with optical microscopy using samples placed between a cover slip and a glass slide separated by a 0.5 mm thick silicone isolator. The organic solvent was so volatile that a significant amount evaporated in open air, resulting in destabilization of the double emulsions. Thus, evaporation was performed in many experiments inside a covered silicone isolator to suppress the evaporation rate. The polymersomes were also be formed by evaporating the organic solvent in a gently stirred glass vial.

Microscopic observations. Bright-field, phase-contrast and fluorescence images were obtained with 10×, 20×, 40×, and 60× objectives at room temperature using an inverted microscope (Leica, DMIRBE), an inverted fluorescence microscope (Leica, DMIRB) or a upright fluorescence microscope (Leica, DMRX) equipped with a high speed camera (Phantom, V5, V7 or V9) or a digital camera (QImaging, QICAM 12-bit). All double emulsion generation processes were monitored with the microscope using a high speed camera. The formation of polymersomes from double emulsions and the resulting polymersomes were imaged with a digital camera. The size distribution of the polymersomes was obtained by measuring the size of at least 300 polymersomes from an optical microscope image

Interfacial tension measurements. Characteristic interfacial tensions were measured by forming a pendant drop of the denser phase at the tip of a blunt stainless steel needle (McMaster-Carr, 20 Gauge) immersed in the other phase and fitting the Laplace equation to the measured drop shape.

### Example 2

Liposomes or vesicles are phospholipid bilayer membranes which surround aqueous compartments. They are promising delivery vehicles for drugs, enzymes, and gases, and bioreactors for biomedical applications. Since phospholipids are an integral component of biological membranes, phospholipid vesicles also provide ideal platforms for the study of the physical properties of biomembranes. Conventional vesicle formation techniques such as hydration and electroformation rely on the self-assembly of phospholipids in an aqueous environment under shear and electric field, respectively. Due to the random nature of the bilayer folding, these methods typically lead to the formation of vesicles that are non-uniform in both size and shape. Moreover, the encapsulation efficiency of these processes is quite low, generally less than 35%.

This example illustrates a technique for forming phospholipid vesicles using monodisperse double emulsions with a core-shell structure as templates. Because of the resemblance of core-shell structures to vesicular structures, techniques that rely on double emulsion templates should be robust and straightforward. In this approach, phospholipids were dissolved in a mixture of volatile organic solvents that is immiscible with aqueous phases. The phospholipid solution formed the shell of water-in-oil-in-water (W/O/W) double emulsions. The phospholipid-stabilized W/O/W double emulsion drops were used as templates to direct the formation of phospholipid vesicles by removing the solvent in oil phase through evaporation, as illustrated in FIG. 10. This example illustrates strategies to improve the stability of phospholipid vesicles during solvent removal. This technique can be used to create phospholipid vesicles with different composition while maintaining high size uniformity and encapsulation efficiency.

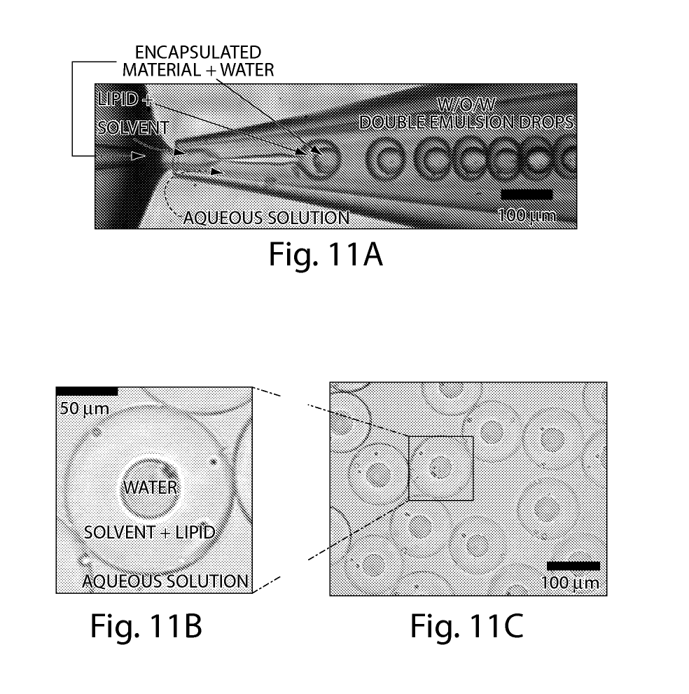

Monodisperse double emulsions were generated with a glass microcapillary microfluidic device that combined a co-flow and a flow focusing geometry shown in FIG. 11A. The inner phase (water, in this example) was an aqueous solution of encapsulant while the outer phase was an aqueous solution of polyvinyl alcohol (PVA) and glycerol. The middle phase was a solution of phospholipids (lipid) dissolved in a mixture of toluene and chloroform (the solvent). Hydrodynamically focused inner and middle fluid streams broke up at the orifice of the collection tube to form monodisperse W/O/W double emulsion drops, as shown in FIG. 11A. In particular, this figure shows the formation of a phospholipid-stabilized W/O/W double emulsion in a glass microcapillary device. A typical droplet generation frequency was about 500 Hz. The overall size and the thickness of the shell of the double emulsions could be adjusted by tuning the flow rates of each fluid phase and the diameters of each capillary in the device. The uniformity in size and shape of the collected double emulsion drops, shown in FIG. 11B, made them ideal templates for the generation of uniform phospholipid vesicles. This figure shows an optical micrograph of the double emulsion collected. The double emulsion drops had an aqueous core surrounded by a solvent shell containing phospholipid. In the absence of phospholipids, the double emulsions were somewhat unstable, suggesting that phospholipids adsorb at the W/O and O/W interfaces and stabilize the structures.

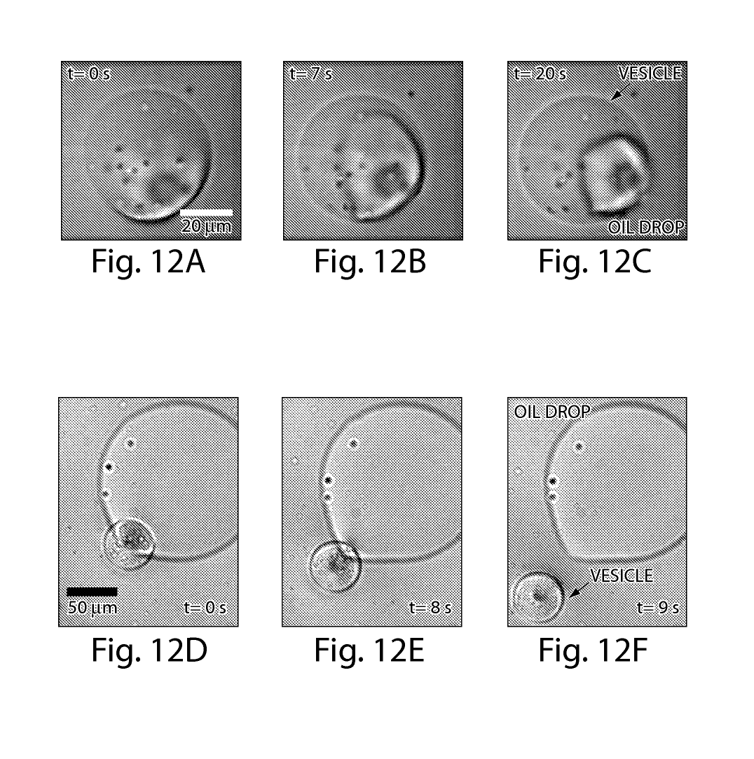

Phospholipid vesicles were obtained from the double emulsions by removing the solvent from the hydrophobic layer of W/O/W double emulsions (FIG. 10). A mixture of volatile organic solvents, toluene and chloroform, was used to facilitate phospholipid dissolution and subsequent solvent evaporation. As the solvent layer gets thinner during evaporation, the phospholipids were concentrated and then forced to arrange on the double emulsion templates, thereby forming vesicles. At the later stage of evaporation, the remaining solvent containing the excess phospholipids accumulated on one side of the vesicle, as shown in the top panel of FIG. 12. Such a dewetting phenomenon has also been observed when amphiphilic diblock copolymers are used for the generation of polymersomes from double emulsions, as discussed above. The depletion force generated by excess phospholipid molecules in the solvent was believed to induce the dewetting.

FIGS. 12A-12C show vesicle formation through solvent drying on the vesicle surface. Excess phospholipid is concentrated in the remaining oil drop attached to the resulting vesicle. FIGS. 12D-12F show the release of vesicle from a double emulsion drop pinned on a glass slide. The oil drop that contained excess phospholipids remained on the glass slide. Fluorescently labeled latex particles, which were added to the inner aqueous phase during double emulsion formation, were also encapsulated in the vesicles.

The vesicles sometimes destabilized and ruptured during the evaporation process. This could be avoided or reduced by slowing solvent evaporation of the organic solvent.

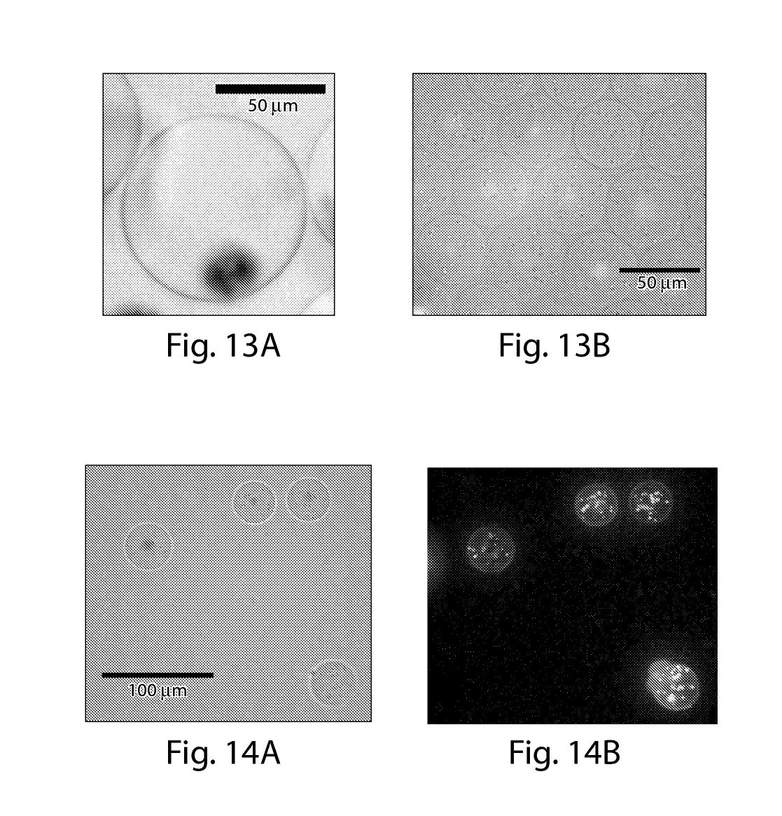

In some cases, a loosely sealed container was used to slow evaporation. The vesicles also became more stable against rupture when the evaporation step is carried out in highly concentrated glycerol solutions (typically above 80 wt %). It is believed that glycerol plays an important role in reducing the line tension incurred in the solvent removal step. After the complete removal of the solvent, the excess phospholipids remained on the vesicle, leaving a thicker patch, as seen as a dark spot in FIG. 13A. The size of this patch was minimized when the amount of excess phospholipid in the oil phase was reduced by reducing the phospholipid concentration in the middle fluid and/or by forming a thinner shell when generating the double emulsion. FIG. 13A is an optical micrograph of a DPPC:DPPS (10:1 w/w) vesicle formed by solvent drying. Excess phospholipids remained on the vesicle forming the dark spot after drying.

Phospholipid vesicles could also be formed through another mechanism. When the double emulsion droplets wet the substrate, they can become pinned to it, and the inner drops can be released as vesicles into the continuous phase. Upon release of the inner drops, the middle organic solvent layer remained pinned to the substrate, as shown in FIG. 12B. This process resembles a method where phospholipid stabilized-water droplets are formed in oil and subsequently transported through an oil/water interface that is covered with a monolayer of phospholipids, resulting in the generation of vesicles. In this case, the inner drops of the pinned double emulsion, stabilized by phospholipids, moved across the interface between the oil and the continuous aqueous phase. Phospholipids adsorbed at this water-oil interface stabilized the escaping inner drop by completing the bilayers. This route to phospholipid vesicle generation offers a simple and effective way of obtaining homogeneous vesicles if the double emulsions can be controllably pinned on a substrate.

An array of monodisperse phospholipid vesicles that have been formed through this second mechanism are shown in FIG. 13B, which illustrates an optical micrograph of an array of homogeneous POPC vesicles, encapsulating 1 micrometer fluorescent latex particles that have been added to the inner aqueous phase. Using the same approach, vesicles have been generated using a variety of phospholipids including both saturated (e.g., DPPC, DMPC, or DSPC) and unsaturated (e.g., DOPC or POPC) phosphocholines used alone or mixed with a phospho-L-serine (DPPS). The typical size of the vesicles ranges from 20 micrometers to 150 micrometers, a size where monodisperse vesicles can be difficult to obtain otherwise.

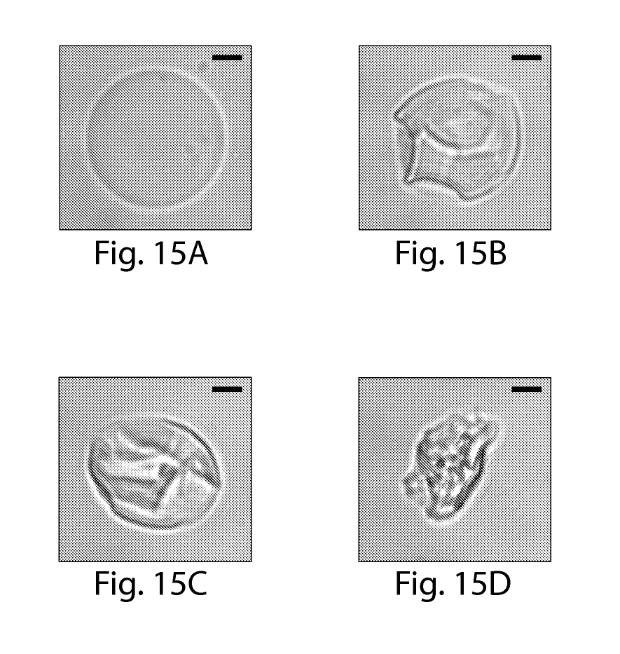

To demonstrate the high encapsulation efficiency of our approach, 1 micrometer yellow-green fluorescent latex microspheres were encapsulated inside phospholipid membranes, which were labeled with a small amount (0.02 mol %) of Texas Red-labeled 1,2-dihexanoyl-sn-glycero-3-phosphoethanolamine (TR-DHPE). Optical and fluorescence microscopy images of four DPPC vesicles encapsulating microspheres are shown in FIGS. 14A and 14B. These figures show that very few microspheres were observed in the continuous phase, thus showing that the high encapsulation efficiency of the double emulsion generation stage was retained even after the emulsion drops were converted to vesicles. In addition, FIG. 14A is an optical micrograph of yellow-green fluorescent latex microspheres encapsulated inside DPPC vesicles stained with 0.02 mol % of Texas red labeled DHPE for visualization. FIG. 14B shows an overlay of two fluorescent images of the same vesicles as in FIG. 14A. The microspheres remain encapsulated within the vesicles.

In conclusion, this example illustrates one general method for fabricating monodisperse phospholipid vesicles using controlled double emulsions as templates. Our simple and versatile technique offers a novel route to generate monodisperse phospholipid vesicles with high encapsulation efficiency for biomedical applications and for fundamental studies of biomembrane physics.

Details regarding the above experiments follow. The inner phase of the water-in-oil-in-water (W/O/W) double emulsion droplets was made of 0-5 wt % poly(vinyl alcohol) (PVA; M_{w}: 13000-23000 g·mol⁻¹, 87-89% hydrolyzed, Sigma-Aldrich Co.) and ˜0.02 wt % 1 micrometer yellow-green sulfate-modified microspheres (Fluosphere, Invitrogen, Inc.). Unless otherwise noted, the middle organic phase was 5-10 mg·mL⁻¹ lipids with 0.02 mol % Texas red labeled 1,2-dihexanoyl-sn-glycero-3-phosphoethanolamine (TR-DHPE) for fluorescent visualization in an organic solvent mixture of toluene (EMD Chemicals, Inc.) and chloroform (Mallinckrodt Chemicals, Inc.) in 1.8-to-1 volume ratio. The experiments were conducted with the following lipids: 1,2-dipalmitoyl-sn-glycero-3-phosphocholine (DPPC), 1,2-dimyristoyl-sn-glycero-3-phosphocholine (DMPC), 1,2-dioleoyl-sn-glycero-3-phosphocholine (DOPC), 1-palmitoyl-2-oleoyl-sn-glyceo-3-phoscholine (POPC), 1,2-distearoyl-sn-glycero-3-phosphocholine (DSPC), 1,2-diacyl-sn-glycero-3-phospho-L-serine (DPPS) and Texas red labeled 1,2-dihexanoyl-sn-glycero-3-phosphoethanolamine (TR-DHPE). All lipids were purchased in powder form from Avanti Polar Lipids, Inc. The outer phase was either a 10 wt % poly(vinyl alcohol) (PVA; M_{w}: 13000-23000 g·mol⁻¹, 87-89% hydrolyzed) solution or a 40 vol % glycerol and 2 wt % PVA solution. The solutions and solvents were all filtered before introduction into glass microcapillary devices. Water with a resistivity of 18.2 megohm cm⁻¹ was acquired from a Millipore Milli-Q system.

Monodisperse W/O/W double emulsions were prepared in glass microcapillary devices. The round capillaries, with inner and outer diameters of 0.58 mm and 1.0 mm, were purchased from World Precision Instruments, Inc. and tapered to desired diameters with a micropipette puller (P-97, Sutter Instrument, Inc.) and a microforge (Narishige International USA, Inc.). The tapered round capillaries were fitted into square capillaries (Atlantic International Technology, Inc.) with an inner dimension of 1.0 mm for alignment. The outer radii, Rₒ, of the double emulsions varied from 60 to 100 micrometers, while the inner radii, Rᵢ, varied from 40 to 60 micrometers. These values were controlled by the size of the capillaries used and the flow rates of the different phases. A typical set of flow rates for the outer, middle and inner phase was 3500 microliters/hr, 800 microliters/hr and 220 microliters/hr, and the droplet generation frequency was about 500 Hz. The formation of lipid vesicles was monitored via optical microscopy for samples placed between a cover slip and a glass slide separated by a 0.5 mm thick silicone isolator (Invitrogen, Inc.).

Bright-field, phase-contrast and fluorescence images were obtained with 5×, 10×, 20×, and 40× objectives at room temperature using a inverted fluorescence microscope (Leica, DMIRB or DMIRBE) or a upright fluorescence microscope (Leica, DMRX) equipped with a high speed camera (Phantom, V5, V7 or V9) or a digital camera (QImaging, QICAM 12-bit). All double emulsion generation processes were monitored with the microscope using a high speed camera. The process of lipid vesicle formation from double emulsions and the resulting lipid vesicles were imaged with a digital camera.

### Example 3

Colloidosomes are microcapsules whose shell comprise colloidal particles. Their physical properties such as permeability, mechanical strength, or biocompatibility can be controlled through the proper choice of colloids and preparation conditions for their assembly. The ability to control their physical properties makes colloidosomes attractive structures for encapsulation and controlled release of materials ranging from fragrances and active ingredients to molecules produced by living cells.

This example demonstrates that nanoparticle colloidosomes with selective permeability can be prepared from monodisperse double emulsions as templates. Monodisperse water-in-oil-in-water (W/O/W) double emulsions with a core-shell geometry were generated using glass capillary microfluidic devices. Hydrophobic silica nanoparticles dispersed in the oil shell stabilized the droplets and ultimately become the colloidosome shells upon removal of the oil solvent. The size of these double emulsions, and thus the dimensions of the resulting colloidosomes, could be precisely tuned by independently controlling the flow rates of each fluid phase. Unlike the colloidosomes that are templated by water droplets in a continuous phase of oil, these colloidosomes were generated directly in a continuous phase of water; thus, there was no need to transfer the colloidosomes from an oil to an aqueous phase. Also, by incorporating different materials into the oil phase, it was possible to prepare composite colloidosomes. The thickness of the colloidosome shells, which is a critical parameter determining the mechanical strength and permeability of colloidosomes, could be controlled by changing the dimension of the double emulsion templates. These nanoparticle colloidosomes have selective permeability to molecules of different sizes. The permeability of low molecular weight molecules was investigated using the fluorescence recovery after photobleaching (FRAP) method. This approach to prepare colloidosomes from W/O/W double emulsion templates provided a robust and general method to create monodisperse semi-permeable nanoparticle colloidosomes with precisely tuned structure and composition.

The microfluidic device used in this example combined a flow focusing and co-flowing geometry, as schematically illustrated in FIG. 17A. This geometry resulted in hydrodynamic flow focusing of three different fluid streams at the orifice of the collection tube and leads to the formation of double emulsions. Water was used as the inner and outer phases and a volatile organic solvent such as toluene or a mixture of toluene and chloroform was used as the middle phase. The double emulsions were stabilized by hydrophobic silica (SiO₂) nanoparticles, which were dispersed in the oil phase without addition of surfactant. Without the nanoparticles, the double emulsions generated in the microcapillary devices did not appear to be stable. The double emulsions were stabilized by nanoparticles which adsorb to the two oil/water interfaces. After the nanoparticle stabilized double emulsions were collected, the oil phase was removed by evaporation, leading to the formation of nanoparticle colloidosomes through dense packing of nanoparticles as shown schematically in FIG. 17B.

The double emulsions generated from microcapillary devices appeared to be substantially monodisperse, as evidenced by the hexagonal close packing of the drops, illustrated by optical and fluorescence microscopy images in FIGS. 17C and 17D, respectively. These double emulsions encapsulated molecules in the inner aqueous phase with near 100% efficiency. Such high encapsulation efficiency is possible since the drop formation process does not allow the inner aqueous phase to come in contact with the outer aqueous phase (FIG. 17A). Thus, as long as the encapsulated materials cannot permeate through the oil phase, essentially all of the molecules and materials could be retained within the interior of the drops. To illustrate this, 250 micrograms/mL dextran-labeled with fluorescein isothiocyanate (FITC-dextran, MW=70 k) was dissolved in the inner aqueous phase; it could not be detected in the continuous outer phase, as seen in fluorescence microscope image in FIG. 17D.

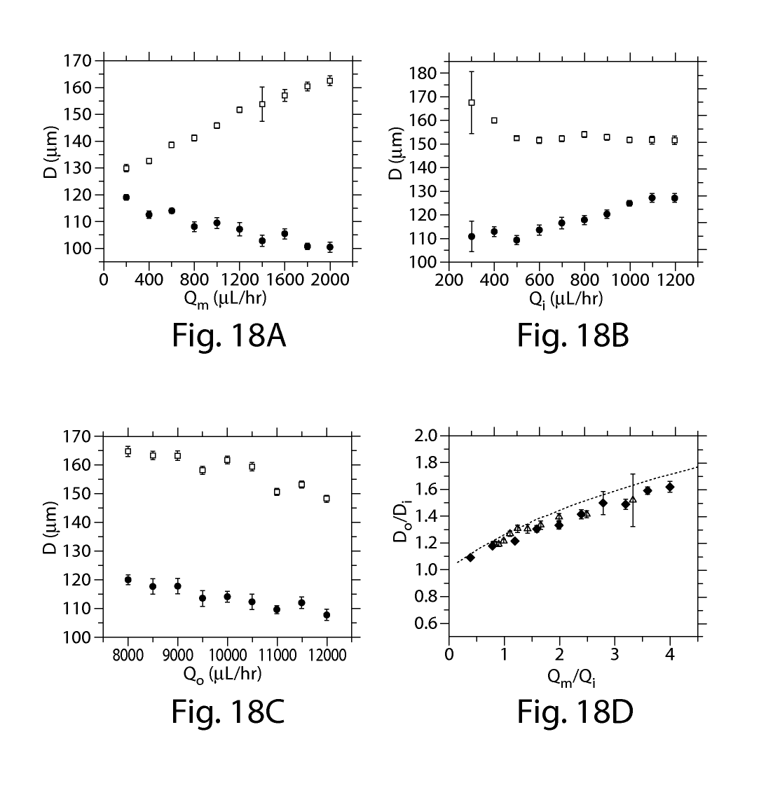

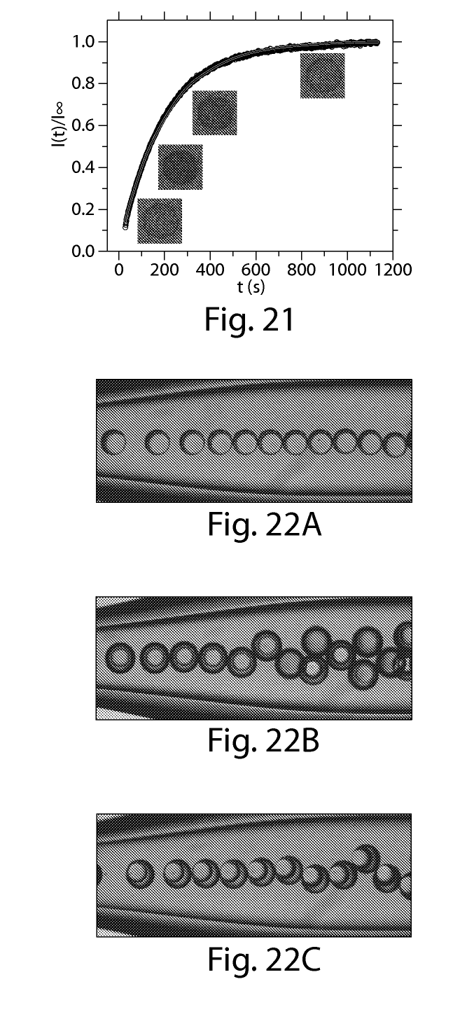

One major advantage of using microcapillary devices to create the templates for colloidosome generation is in the precise control over the dimensions of the double emulsions; the size of inner drop (Dᵢ) and outer drop (Dₒ), thus the thickness of oil shell (H=(Dₒ−Dᵢ)/2), can be precisely and independently tuned by changing the flow rates (Q) of each phase. For example, increasing the flow rate of the middle phase (Qₘ) leads to the formation of drops with larger H and smaller Dᵢ as illustrated in FIG. 18A. By contrast, increasing the flow rate of the inner phase (Qᵢ) results in formation of drops with larger Dᵢ and smaller H as shown in FIG. 18B. Drops with smaller Dₒ and Dᵢ, but with an approximately constant H, can be generated by increasing Qₒ (flow rate of the outer phase) as shown in FIG. 18C (see also FIG. 22 for images of double emulsions with different dimensions). Flow rates in each image in FIG. 22 are summarized in Table 1. The width of each figure is 1580 micrometers.

TABLE 1
Flow rates of each fluid phase applied to generate double
emulsions in FIG. 22. All units are in μL/hr.

| | Qᵢ | Qₘ | Qₒ |
|---|---|---|---|
| (a) | 10000 | 400 | 500 |
| (b) | 10000 | 2000 | 500 |
| (c) | 10000 | 1000 | 400 |
| (d) | 10000 | 1000 | 1200 |
| (e) | 8000 | 1000 | 500 |
| (f) | 12000 | 1000 | 500 |

The results from FIGS. 18A and 18B are summarized by plotting Dₒ/Dᵢ as a function of Qₘ/Qᵢ in FIG. 18D and show good agreement with the predicted values (dotted line in FIG. 18D) estimated from:

$$\frac{{D}_{o}}{{D}_{i}} = {( 1 + \frac{{Q}_{m}}{{Q}_{i}} )}^{\frac{1}{3}} . \qquad ( 1 )$$

The high degree of control over the drop dimensions afforded by this approach allowed the fabrication of colloidosomes with precisely tuned structure.

FIG. 18 thus shows the effect of flow rates (Q) on the size of double emulsions. In FIG. 18A, the flow rate of oil phase (Qₘ) was varied while the flow rates of inner (Qᵢ) and outer phases (Qₒ) were kept constant at 500 and 10,000 microliters/hr, respectively. In FIG. 18B, Qᵢ was varied while Qₘ and Qₒ were kept constant at 1,000 and 10,000 microliters/hr, respectively. In FIG. 18C, Qₒ was varied while Qₘ and Qᵢ were kept constant at 1,000 and 500 microliters/h, respectively. Open squares and closed circles represent the diameters (D) of outer and inner drops, respectively, in FIGS. 18A-18C. FIG. 18D is a plot of size ratio of outer to inner drop (Dₒ/Dᵢ) versus flow rate ratio of middle to inner phase (Qₘ/Qᵢ). The dotted line represents predicted values of Dₒ/Dᵢ based on Equation 1. Closed diamonds and open triangles in FIG. 18D are data from FIGS. 18A and 18B, respectively. In all cases, the following solutions were used for each phase: outer phase=2 wt % PVA in water, middle phase=7.5 wt % silica nanoparticle in toluene and inner phase=2 wt % PVA solution.

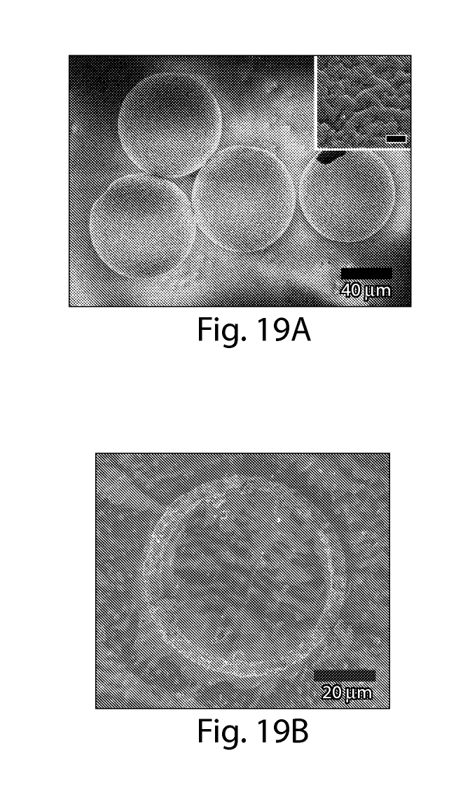

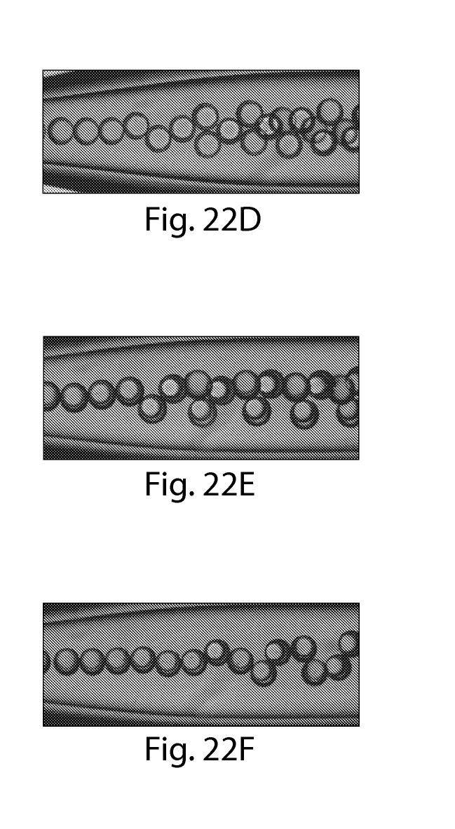

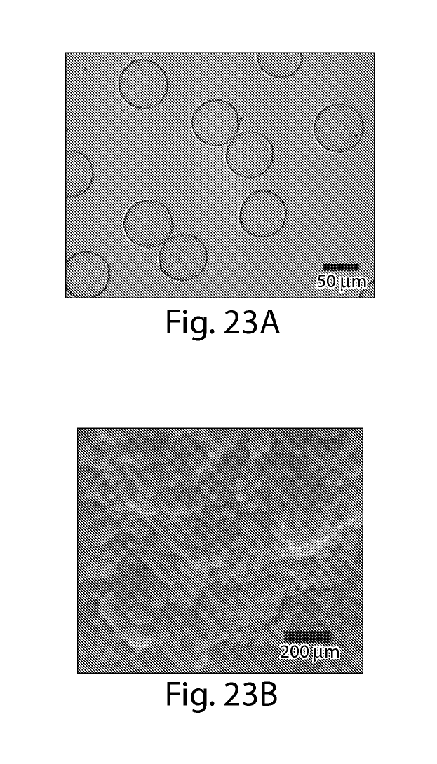

Once the double emulsions were collected from the glass microcapillary device, nanoparticle colloidosomes are formed by removing the oil phase through evaporation (FIG. 17B). A scanning electron microscopy (SEM) image of monodisperse colloidosomes prepared by evaporating toluene is shown in FIG. 19A (see FIG. 23 for an optical microscope image of colloidosomes). The inset is a high magnification image of colloidosome surface (scale bar=600 nm). While colloidosomes with thin shells tended to collapse upon drying, those with thicker shells are able to structurally withstand the evaporation process and retained their spherical shape (FIG. 19A). Close inspection of the colloidosome surfaces revealed wrinkles that resemble the herringbone buckling patterns observed in equi-biaxially compressed stiff thin films atop elastomeric substrates. These wrinkles developed during evaporation of the oil phase. It appears that the nanoparticles adsorbed to the water-toluene interface to form a two-dimensional network and buckled during evaporation and shrinkage of the oil phase.

This approach provides a technique to independently control the thickness of the shell of the colloidosomes; this may be important in tuning their mechanical strength and permeability. The thickness and structure of colloidosome shells were observed by freeze-fracture cryogenic-scanning electron microscopy (cryo-SEM), which revealed that the shell thickness was uniform and appeared defect free, as illustrated in FIG. 19B. Colloidosomes could be created with shell thicknesses ranging from 100 nm to 10 micrometers by controlling the dimension of the double emulsions and the volume fraction of nanoparticles in the oil phase. A high magnification cryo-SEM image shows that the nanoparticles are randomly and densely packed to form the shell of the colloidosomes.

In addition to nanoparticle colloidosomes, this approach allowed the preparation of multicomponent colloidosomes, or composite microcapsules. For example, by dissolving poly(D,L-lactic acid) (PLA), which is a biodegradable polymer, in the oil phase containing hydrophobic silica nanoparticles, PLA/SiO₂ composite microcapsules could be prepared, as seen in FIG. 19C, which is an SEM image of poly(DL-lactic acid) (PLA)/SiO₂ composite capsules dried on a substrate. The thickness of the composite capsule shell was approximately 200 nm as shown in the inset of FIG. 19C (scale bar=500 nm); this is in agreement with the estimate of 220 nm based on the volume fraction of solid materials (10 vol %) in the oil phase. Magnetically responsive composite colloidosomes can also be prepared by suspending Fe₃O₄ magnetic nanoparticles along with hydrophobic silica nanoparticles in the oil phase. These magnetic colloidosomes could be separated from the solution using a magnetic field as shown in FIG. 19D (showing magnetic separation of 10 nm Fe₃O₄ nanoparticle containing colloidosomes). These examples demonstrate that it is straightforward to fabricate composite colloidosomes with precisely tuned composition; this is difficult to achieve using other methods.

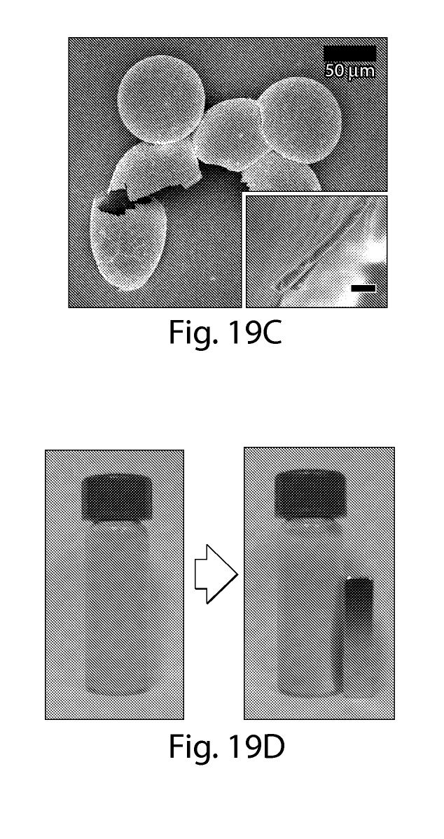

Since colloidosomes are made from colloidal particles, their shells are intrinsically porous due to the presence of interstitial voids between the packed particles. The selective permeability of these colloidosomes was demonstrated by exposing them to aqueous solutions of fluorescence probes with different molecular weights. The permeation of fluorescence probes into the interior of the colloidosomes is detected by confocal laser scanning microscopy (CLSM). Calcein, a low molecular weight (Mw=622.55) fluorescent molecule, freely diffused into the interior of SiO₂ nanoparticle colloidosomes as shown in FIG. 20A (FIGS. 20A-20C each show confocal laser scanning microscope images; in all cases, the images were taken ˜30 min after the addition of probe molecules). By contrast, dextran labeled with fluorescein isothiocyanate (FITC-dextran), a high molecular weight polymer (Mw˜2,000,000), did not diffuse into the interior of the colloidosomes (FIG. 20B). The striking difference in the permeability appeared to be due to size exclusion and demonstrated the selective permeability of these colloidosomes. The pore size of randomly closed packed spheres is approximately 10% of the radius. Therefore, calcein, whose size is less than 1 nm, could apparently diffuse into the colloidosomes without much resistance as the size of nanoparticles used for their fabrication was 10˜20 nm. By contrast, it was very difficult for the high molecular weight dextran, whose radius of gyration is ˜40 nm, to diffuse through the shell of the colloidosomes.

The diffusion of calcein could, however, be prevented or reduced by incorporating a polymer, such as PLA, into the colloidosome structures as illustrated by colloidosomes with dark interiors in FIG. 20C. These composite colloidosomes remained impermeable to calcein at least for 24 hr. The polymer apparently filled the interstices between the nanoparticles making the composite capsules essentially impermeable. These results demonstrated that the shells of nanoparticle colloidosomes were porous and that colloidosomes exhibit selective permeability; moreover, by incorporating polymers into the colloidosomes, the permeability of small molecular weight molecules could be reduced. The size of the pores in the colloidosome shells was proportional to the size of nanoparticles used; therefore, the selectivity of the colloidosomes could be controlled by changing the size of the nanoparticles.

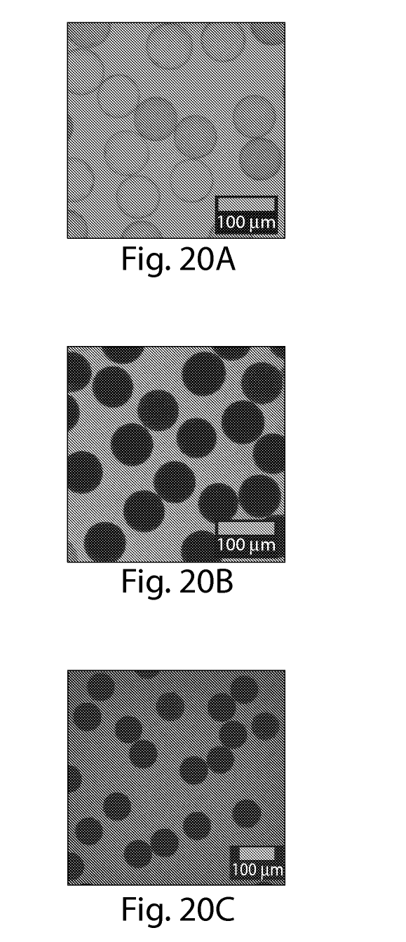

Quantitative information on the permeability of colloidosomes is important for a number of applications including controlled release of fragrances, pesticides, or pharmaceuticals. Fluorescence recovery after photobleaching (FRAP) was used to measure the permeability of a low molecular weight probe, 5(6)-carboxyfluorescein (CF). CF was allowed to permeate into the colloidosomes and then the laser was focused in the interior region of colloidosome, photobleaching the CF that was trapped in the interior. The gradual recovery of fluorescence as a function of time due to the diffusion of unbleached “fresh” probes into the colloidosome is seen in FIG. 21. The temporal evolution of the recovery of fluorescence intensity within a capsule can be described by:

$$\frac{I ( t )}{{I}_{∞}} = 1 - {e}^{- A t} \qquad ( 2 )$$

where, A=3P/r. P is the permeability of the probe through the colloidosome shell and r is the radius of the colloidosome. I(t) and I_{∞} represent the intensity of fluorescence probe within colloidosomes at time t and t→∞, respectively, assuming that complete photobleaching is achieved at t=0. Using Equation 2 (the curve in this figure), the permeability of CF across nanoparticle colloidosome shell was determined to be 0.062±0.028 μm/s. Since diffusivity is the product of permeability (P) and the thickness of the shell, the value of permeability could be converted to the diffusion coefficient of CF molecules across the nanoparticle colloidosome; thus, the diffusion coefficient of the probe was estimated to be 3.7×10⁻² μm²/s.

FIG. 23A illustrates optical microscopy image of colloidosomes suspended in water after removal of solvent. FIG. 23B illustrates high magnification freeze-fracture cryo-SEM image of colloidosome shell showing densely packed nanoparticles.

Thus, this example demonstrates that semipermeable colloidosomes comprising nanoparticles and other materials including polymers can be prepared from water-in-oil-in-water (W/O/W) double emulsions. This approach provides a general and robust method to generate monodisperse nanoparticle colloidosomes and composite microcapsules. By controlling the size of nanoparticles, it is possible to control the selectivity as well as the permeability of nanoparticle colloidosomes making them attractive systems to encapsulate active ingredients, drugs, or food ingredients for applications in controlled release and drug delivery.

Following are additional details regarding the experiments discussed in this example. Glass microcapillaries were purchased from World Precision Instruments, Inc. and Atlantic International Technologies, Inc. Hydrophobic silica nanoparticles suspended in toluene were provided by Nissan Chemical Inc. (Japan). Toluene, calcein, 5(6)-carboxyfluorescein (CF), FITC-labeled dextran (Mw˜2,000,000 and 70,000) and polyvinyl alcohol (PVA; 89˜92% hydrolyzed, Mw˜70,000) were obtained from Sigma Aldrich. Poly(D,L-lactic acid) (PLA; Mw˜6,000˜16,000, polydispersity index (PDI)=1.8) was obtained from Polysciences. 10 nm magnetic nanoparticles suspended in toluene were purchased from NN Labs, LLC. Chemicals were used as received without further purification.

Microcapillary device fabrication and generation of double emulsions. Briefly, cylindrical glass capillary tubes with an outer diameter of 1 mm and inner diameter of 580 micrometers were pulled using a Sutter Flaming/Brown micropipette puller. The dimension of tapered orifices was adjusted using a microforge (Narishige, Japan). Typical dimensions of orifice for inner fluid and collection were 10˜50 micrometers and 30˜500 micrometers, respectively. The orifice sizes could be adjusted with the puller and the microforge to control the dimensions of double emulsions. The glass microcapillary tubes for inner fluid and collection were fitted into square capillary tubes that had an inner dimension of 1 mm. By using the cylindrical capillaries whose outer diameter are the same as the inner dimension of the square tubes, a good alignment could be easily achieved to form a coaxial geometry. The distance between the tubes for inner fluid and collection was adjusted to be 30˜150 micrometers (FIG. 18A). A transparent epoxy resin was used to seal the tubes where required. Solutions were delivered to the microfluidic device through polyethylene tubing (Scientific Commodities) attached to syringes (Hamilton Gastight or SGE) that were driven by positive displacement syringe pumps (Harvard Apparatus, PHD 2000 series). The drop formation was monitored with a high-speed camera (Vision Research) attached to an inverted microscope.

For the generation of W/O/W double emulsions, three fluid phases were delivered to the glass microcapillary devices. The outer aqueous phase comprised 0.2˜2 wt % PVA solution and the inner aqueous phase comprised 0˜2 wt % PVA solution. The middle phase typically was about 7.5 wt % hydrophobic silica nanoparticles suspended in toluene. The concentration of nanoparticles in the middle phase was varied between 3 and 22 wt %. PLA/SiO₂ nanoparticle composite microcapsules were prepared by adding PLA and silica nanoparticles to toluene at a concentration of 50 mg/ml and 7.5 wt %, respectively. Magnetically responsive colloidosomes were prepared by mixing silica nanoparticle suspension (45 wt % in toluene), magnetic nanoparticle suspension (10 nm in diameter, 2 mg/ml in toluene) and toluene in a 1:4:1 volumetric ratio.

To convert double emulsion droplets to nanoparticle colloidosomes, the emulsion was exposed to vacuum overnight. The nanoparticle colloidosomes were then washed with a copious amount of de-ionized water to remove the remaining oil phase. Scanning electron microscopy was performed on a Zeiss Ultra55 field emission scanning electron microscope (FESEM) at an acceleration voltage of 5 kV. Samples were coated with approximately 5˜10 nm of gold. Freeze-fracture cryo-SEM was performed on a Dual Beam 235 Focused Ion Beam (FIB)-SEM at an acceleration voltage of 5 kV. A small aliquot of sample was placed on a sample stub and was plunged into liquid nitrogen. The frozen sample was fractured using a sharp blade and coated with a thin layer of Au before imaging.

Permeability measurement via fluorescence recovery after photobleaching (FRAP). A small volume (˜50 microliters) of NP colloidosome suspension was place in an elastomer isolation chamber atop a glass coverslide. The colloidosomes were allowed to sediment to the bottom of the chamber for 30 min before FRAP experiments. FRAP was performed using Leica TCS SP5 confocal microscope. Ar laser at a wavelength of 488 nm was used at maximum intensity to photobleach the dyes, and the recovery was observed at 1% of the bleaching intensity at 1-2 sec intervals.

### Example 4

This example illustrates the formation of polymersomes by directing the assembly of amphiphilic diblock copolymers using double emulsion drops as templates. As the volatile solvent evaporates, the concentration of the diblock copolymer increases in the shell layer. Eventually, the double emulsion drops undergo a dewetting transition to form acorn-shaped drops. One side of the drops contains the solvent with the diblock copolymer whereas the opposite side is a vesicular compartment where the aqueous core is separated from the surroundings by a thin layer of diblock copolymers. The walls typically are a bilayer of the amphiphilic diblock copolymers and have sub-micron thickness. Since the inside of the vesicle wall is made up of the hydrophobic block, it may be an ideal location for encapsulating the drugs that are typically hydrophobic.

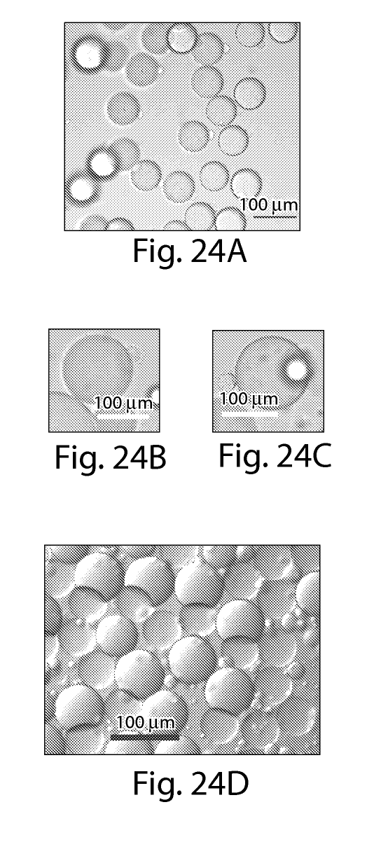

In some cases, PEG-b-PLA polymersomes can be formed using a solvent mixture of chloroform and toluene. While chloroform acts as a “good” solvent for dissolving the diblock copolymers, the role of toluene was not entirely clear. One possible role of the toluene is to reduce the solubility of the solvent mixture for the diblock copolymers. To address the role of toluene, the fabrication process was repeated using other solvents such as silicone oil with different viscosities and hexane, while keeping chloroform as the solvent for the diblock copolymers. It was observed that the dewetting double emulsion drops were stable on at a limited range of good solvent concentration. When the volume fractions of chloroform was below 40% for silicone oils with viscosities of 0.65 cSt and 1 c St, or below 40% for hexane, the dewetted double emulsion drops remained stable and polymersomes could be formed, as shown in FIG. 24. In particular, FIG. 24A illustrates the formation of polymersomes from a solvent mixture of chloroform and 1 cSt poly(dimethyl siloxane) (PDMS) in a 40:60 volume ratio; FIGS. 24B and 24C illustrate chloroform and 0.65 cSt poly(dimethyl siloxane) (PDMS) in a 40:60 volume ratio, and FIG. 24D illustrates chloroform and hexane in 36:64 volume ratio.

In light of these observations, it is believed that the solvent mixture achieved an optimal solvent quality for this dewetting route towards polymersomes through attractive interactions between the diblock copolymers at the interfaces, which can exist at certain solvent qualities.

By optimizing the volume fractions in the solvent mixture, it is possible to tune the polymersome generation step such that complete dewetting can finish inside the microfluidic devices. In that case, the solvent evaporation step, which is typically time-consuming and leads to polymersomes that are inhomogeneous, can be omitted. This is demonstrated by optimizing the volume fractions of chloroform and hexanes. When the solvent mixture contained about 36% chloroform by volume and 10 mg/mL of PEG(5000)-b-PLA(5000), the double emulsion drops started to and completed dewetting inside the microchannel; polymersomes could be collected at the outlet of the microfluidic device. The collected polymersomes did not have any remaining solvent droplets attached to them. These results suggested that the mechanism for forming polymersomes may be quite general over the use of solvents and that the time-consuming solvent evaporation can be eliminated in some embodiments.

### Example 5

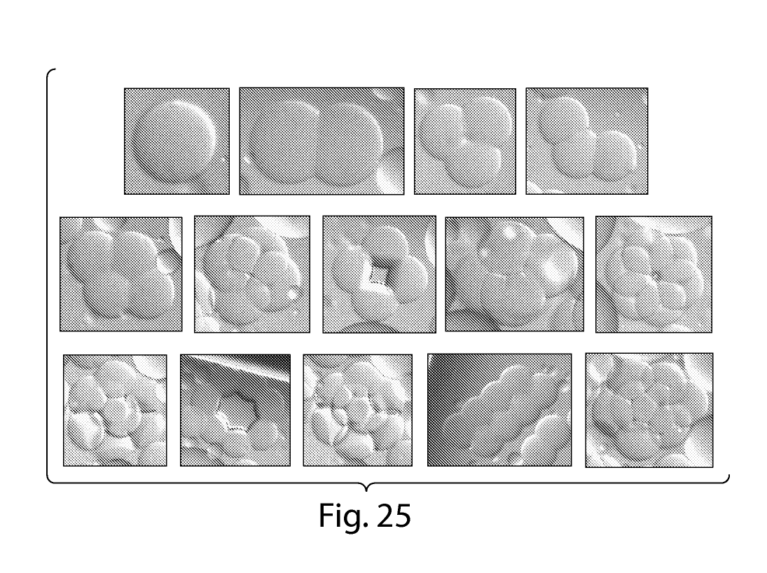

Using the same formulation as in Example 4, multi-compartment polymersomes were formed, as shown in FIG. 25, by generating multiple inner droplets in the double emulsion formation stage. These multi-compartment polymersomes were formed using a middle phase of 10 mg/mL of PEG(5000)-b-PLA(5000) in a mixture of chloroform and hexane in volume ratio of 36 to 64. With microfluidics, controlled number of inner drops could be reliably generated. This allows the possibility of encapsulating different active components in the different inner droplets, eventually leading to encapsulation in different vesicular compartments. Such compartmentalization lead to encapsulation of multiple components within one encapsulating structure. Moreover, if the different components encapsulated interact with each other, the structures allow studies that may have broad implications for cell signaling, and other biochemical reactions.

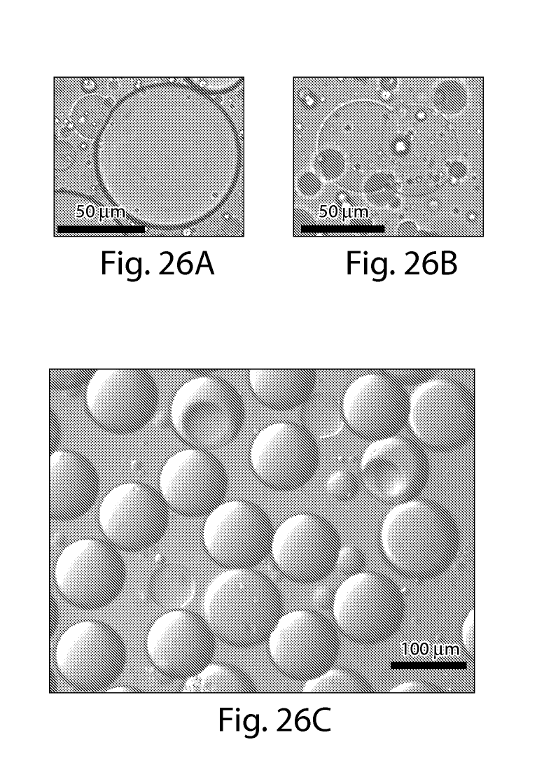

This can also be extended the formation of polymersomes to diblock copolymers that have shorter block lengths. For instance, PEG(3000)-b-PLA(3000) polymersomes were formed as shown in FIGS. 26A-26B (optical micrographs), using a middle phase of 10 mg/mL of PEG(3000)-b-PLA(3000) in a mixture of chloroform and hexane in volume ratio of 36 to 64. Moreover, these methods can also be used to another diblock copolymer of poly(ethylene glycol)-block-poly(caprolactam), PEG(5000)-b-PCL(9000), as shown in FIG. 26C (optical micrograph).

### Example 6

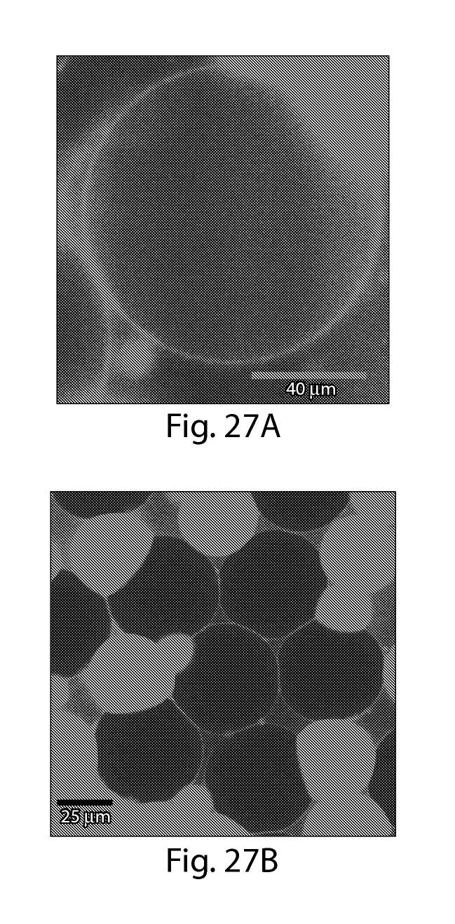

To demonstrate the potential of the encapsulation of actives, such as drugs, in the shell, this example uses DiIC18(3)1,1′-dioctadecyl-3,3,3′,3′-tetramethylindocarbocyanine perchlorate, with a molecular weight of 933.88 g/mol, and Nile red, with a molecular weight of 318.37 g/mol, as model actives, for encapsulation in the shell. Both of these model actives are hydrophobic most drugs of interest; unlike the drugs of interest, these model drugs fluoresces when excited, making them much easier to visualize and verify their presence in the polymersome walls. The polymersomes with these model actives encapsulated are shown in FIG. 27, showing the polymersomes formed with 1 mg/mL DiIC (FIG. 27A) and 1 mg/mL Nile Red (FIG. 27B) added to the middle phase of 10 mg/mL of PEG(5000)-b-PLA(5000) in a mixture of chloroform and hexane in volume ratio of 36 to 64.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

What is claimed is:

1. An article, comprising:
a vesicle comprising a multiblock copolymer, wherein at least one of the blocks of the copolymer is a biodegradable polymer.

2. The article of claim 1, wherein at least one of the blocks of the copolymer comprises poly(lactic acid).

3. The article of claim 1, wherein at least one of the blocks of the copolymer comprises poly(glycolic acid).

4. The article of claim 1, wherein at least one of the blocks of the copolymer comprises poly(ethylene glycol).

5. The article of claim 1, wherein at least one of the blocks of the copolymer comprises poly(caprolactone).

6. The article of claim 1, wherein the vesicle contains a pharmaceutical agent.

7. The article of claim 1, wherein the vesicle is a polymersome.

8. The article of claim 1, wherein the vesicle is a colliodosome.

9. The article of claim 1, wherein the multiblock copolymer is amphiphilic.

10. A method, comprising:
forming a first droplet from a first fluid stream surrounded by a second fluid while the second fluid is surrounded by a third fluid, the second fluid containing a biodegradable polymer; and
reducing the amount of the second fluid in the second fluid droplet.

11. The method of claim 10, wherein the first fluid is miscible in the third fluid.

12. The method of claim 10, wherein the biodegradable polymer is a diblock copolymer, a triblock copolymer and/or a random copolymer.

13. The method of claim 10, wherein the second fluid forms a second fluid droplet surrounding a single droplet of the first fluid.

14. The method of claim 13, wherein greater than about 90% of the second fluid droplets formed contain a single first fluid droplet.

15. The method of claim 10, wherein the second fluid stream forms a droplet around the first droplet.

16. The method of claim 10, wherein the standard deviation of the diameter of the second fluid droplets is less than 10%.

17. The method of claim 10, wherein the second fluid droplet is less than about 200 micrometers in diameter.

18. The method of claim 10, wherein the second fluid is reduced through evaporation.

19. The method of claim 18, wherein the evaporation rate is controlled such that between about 50% and about 90% of the second fluid remains within the second fluid droplet after about 1 day.

20-21. (canceled)

22. A method, comprising:
providing a vesicle comprising a diblock or a triblock copolymer, wherein at least one of the blocks of the copolymer is a biodegradable polymer; and
exposing the vesicle to a change in osmolarity at least sufficient to cause the vesicle to rupture.

23-27. (canceled)
